(19) United States
(12) Patent Application Publication
(10) Pub. No.: US 2010/0048679 A1
(43) Pub. Date: Feb. 25, 2010

(54) POLYNUCLEOTIDE THERAPY

(75) Inventors: Hideki Garren, Palo Alto, CA (US); Michael Leviten, Palo Alto, CA (US); Nanette Solvason, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bayhill Therapeutics, Inc., Palo Alto, CA (US)

(21) Appl. No.: 12/304,628

(22) PCT Filed: Jun. 13, 2007
(86) PCT No.: PCT/US2007/071137

§ 371 Date: Oct. 20, 2009

Related U.S. Application Data
(60) Provisional application No. 60/813,552, filed on Jun. 13, 2006.

(51) Int. Cl. A61K 31/7052; C12N 15/63; A61P 37/06

(52) U.S. Cl. 514/44 R; 435/320.1

(57) ABSTRACT

This invention provides methods of treating an autoimmune disease in a subject associated with one or more self-protein(s), polypeptide(s), or peptide(s) present in the subject non-physiologically comprising administering to the subject: a self-vector comprising an immunosuppressive vector backbone and a polynucleotide encoding the self-protein(s), polypeptide(s) or peptide(s) associated with the autoimmune disease; and a divalent cation at a concentration greater than physiological levels. Administration of the self-vector comprising a polynucleotide encoding the self-protein(s), polypeptide(s) or peptide(s) modulates an immune response to the self-protein(s), polypeptide(s) or peptide(s) expressed from administration of the self-vector This invention further provides a method of treating multiple sclerosis by administering a self-vector comprising a BHT-1 vector backbone, for example, self-vector BHT-3009 encoding human myelin basic protein (MBP). The invention also provides a pharmaceutical composition comprising: a BHT-1 vector backbone and a polynucleotide encoding one or more self-protein(s), polypeptide(s), or peptide(s) associated with an autoimmune disease; and a divalent cation at concentrations greater than physiological levels. This invention further provides a pharmaceutical composition comprising a self-vector comprising a BHT-1 vector backbone, for example, self-vector BHT-3009 encoding human myelin basic protein (MBP), and methods of administering a BHT-1 self-vector, for example BHT-3009, to a subject.

### CROSS-REFERENCES TO RELATED APPLICATIONS

The present application claims the benefit of U.S. Provisional Patent Application No. 60/813,552, the entire disclosure of which is hereby incorporated herein by reference for all purposes.

### STATEMENT AS TO RIGHTS TO INVENTIONS MADE UNDER FEDERALLY SPONSORED RESEARCH OR DEVELOPMENT

NOT APPLICABLE

### BACKGROUND OF THE INVENTION

1. Field of the Invention

The present invention relates to methods and compositions for treating diseases in a subject associated with one or more self-protein(s), -polypeptide(s) or -peptide(s) that are present in the subject and involved in a non-physiological state. The present invention also relates to methods and compositions for preventing diseases in a subject associated with one or more self-protein(s), -polypeptide(s) or -peptide(s) that are present in the subject and involved in a non-physiological state. The invention further relates to the identification of a self-protein(s), -polypeptide(s) or -peptide(s) present in a non-physiological state and associated with a disease. The invention also relates to the administration of a polynucleotide encoding a self-protein(s), -polypeptide(s) or -peptide(s) present in a non-physiological state and associated with a disease. The invention also relates to modulating an immune response to a self-protein(s), -polypeptide(s) or -peptide(s) present in an animal and involved in a non-physiological state and associated with a disease. The invention is more particularly related to the methods and compositions for treating or preventing autoimmune diseases associated with one or more self-protein(s), -polypeptide(s) or -peptide(s) present in the animal in a non-physiological state such as in multiple sclerosis, rheumatoid arthritis, insulin dependent diabetes mellitus, autoimmune uveitis, primary biliary cirrhosis, myasthenia gravis, Sjogren's syndrome, pemphigus vulgaris, scleroderma, pernicious anemia, systemic lupus erythematosus (SLE) and Grave's disease.

### Autoimmune Disease and Modulation of the Immune Response

Autoimmune disease is a disease caused by adaptive immunity that becomes misdirected at healthy cells and/or tissues of the body. Autoimmune disease affects 3% of the U.S. population and likely a similar percentage of the industrialized world population (Jacobson et al., Clin Immunol Immunopathol, 84:223-43 (1997)). Autoimmune diseases are characterized by T and B lymphocytes that aberrantly target self-proteins, -polypeptides, -peptides, and/or other self-molecules causing injury and or malfunction of an organ, tissue, or cell-type within the body (for example, pancreas, brain, thyroid or gastrointestinal tract) to cause the clinical manifestations of the disease (Marrack et al., Nat Med, 7:899-905 (2001)). Autoimmune diseases include diseases that affect specific tissues as well as diseases that can affect multiple tissues. This may, in part, for some diseases depend on whether the autoimmune responses are directed to an antigen confined to a particular tissue or to an antigen that is widely distributed in the body. The characteristic feature of tissue-specific autoimmunity is the selective targeting of a single tissue or individual cell type. Nevertheless, certain autoimmune diseases that target ubiquitous self-proteins can also effect specific tissues. For example, in polymyositis the autoimmune response targets the ubiquitous protein histidyl-tRNA synthetase, yet the clinical manifestations primarily involved are autoimmune destruction of muscle.

The immune system employs a highly complex mechanism designed to generate responses to protect mammals against a variety of foreign pathogens while at the same time preventing responses against self-antigens. In addition to deciding whether to respond (antigen specificity), the immune system must also choose appropriate effector functions to deal with each pathogen (effector specificity). A cell critical in mediating and regulating these effector functions is the CD4⁺ T cell. Furthermore, it is the elaboration of specific cytokines from CD4⁺ T cells that appears to be the major mechanism by which T cells mediate their functions. Thus, characterizing the types of cytokines made by CD4⁺ T cells as well as how their secretion is controlled is extremely important in understanding how the immune response is regulated.

The characterization of cytokine production from long-term mouse CD4+ T cell clones was first published more than 10 years ago (Mosmann et al., J. Immunol, 136:2348-2357 (1986)). In these studies, it was shown that CD4⁺ T cells produced two distinct patterns of cytokine production, which were designated T helper 1 (Th1) and T helper 2 (Th2). Th1 cells were found to exclusively produce interleukin-2 (IL-2), interferon-γ (IFN-γ) and lymphotoxin (LT), while Th2 clones exclusively produced IL-4, IL-5, IL-6, and IL-13 (Cherwinski et al., J. Exp. Med., 169:1229-1244 (1987)). Somewhat later, additional cytokines, IL-9 and IL-10, were isolated from Th2 clones (Van Snick et al., J. Exp. Med., 169:363-368 (1989); Fiorentino et al., J. Exp. Med., 170:2081-2095 (1989)). Finally, additional cytokines, such as IL-3, granulocyte macrophage colony-stimulating factor (GM-CSF), and tumor necrosis factor-α (TNF-α) were found to be secreted by both Th1 and Th2 cells.

Autoimmune disease encompasses a wide spectrum of diseases that can affect many different organs and tissues within the body as outlined in Table 1. See, e.g., Paul W. E. (ed. 2003) Fundamental Immunology (5th Ed.) Lippincott Williams & Wilkins; ISBN-10: 0781735149, ISBN-13: 978-0781735148; Rose and Mackay (eds. 2006) The Autoimmune Diseases (4th ed.) Academic Press, ISBN-10: 0125959613, ISBN-13: 978-0125959612; Erkan, et al. (eds. 2004) The Neurologic Involvement in Systemic Autoimmune Diseases, Volume 3 (Handbook of Systemic Autoimmune Diseases) Elsevier Science, ISBN-10: 0444516514, ISBN-13: 978-0444516510; and Richter, et al. (eds. 2003) Treatment of Autoimmune Disorders, Springer, ISBN-10: 3211837728, ISBN-13: 978-3211837726.

TABLE I

| Primary Organ(s) Targeted | Disease |
|---|---|
| Thyroid | Hashimoto's Disease |
| Thyroid | Primary myxodaema |
| Thyroid | Thyrotoxicosis |
| Stomach | Pernicious anemia |
| Stomach | Atrophic gastritis |
| adrenal glands | Addison's disease |
| pancreatic islets | Insulin dependent diabetes mellitus |
| Kidneys | Goodpasture's syndrome |
| neuromuscular junction | Myasthenia gravis |
| leydig cells | Male infertility |
| Skin | Pemphigus vulgaris |
| Skin | Pemphioid |
| Eyes | Sympathetic ophthalmia |
| Eyes | Phacogenic uveitis |
| Brain | Multiple sclerosis |
| red blood cells | Hemolytic anemia |
| Platelets | Idiopathic thrombocytopenic purpura |
| white blood cells | Idiopathic leukopenia |
| biliary tree | Primary biliary cirrhosis |
| Bowel | Ulcerative colitis |
| Arteries | Atherosclerosis |
| salivary and lacrimal glands | Sjogren's syndrome |
| synovial joints | Rheumatoid arthritis |
| Muscle | Polymyositis |
| muscle and skin | Dermatomyositis |
| Skin | Scleroderma |
| skin, joints, muscle, blood cells | Mixed connective tissue disease |
| clotting factors | Anti-phospholipid disease |
| Skin | Discoid lupus erythematosus |
| skin, joints, kidneys, brain, | Systemic lupus erythematosus (SLE) |
| blood cells | |

Current therapies for human autoimmune disease, include glucocorticoids, cytotoxic agents, and recently developed biological therapeutics. In general, the management of human systemic autoimmune disease is empirical and unsatisfactory. For the most part, broadly immunosuppressive drugs, such as corticosteroids, are used in a wide variety of severe autoimmune and inflammatory disorders. In addition to corticosteroids, other immunosuppressive agents are used in management of the systemic autoimmune diseases. Cyclophosphamide is an alkylating agent that causes profound depletion of both T- and B-lymphocytes and impairment of cell-mediated immunity. Cyclosporine, tacrolimus, and mycophenolate mofetil are natural products with specific properties of T-lymphocyte suppression, and they have been used to treat SLE, RA and, to a limited extent, in vasculitis and myositis. These drugs are associated with significant renal toxicity. Methotrexate is also used as a “second line” agent in RA, with the goal of reducing disease progression. It is also used in polymyositis and other connective-tissue diseases. Other approaches that have been tried include monoclonal antibodies intended to block the action of cytokines or to deplete lymphocytes. (Fox, D. A., Am. J. Med., 99:82-88 (1995).) Treatments for multiple sclerosis (MS) include interferon β and copolymer 1, which reduce relapse rate by 20-30% and only have a modest impact on disease progression. MS is also treated with immunosuppressive agents including methylprednisolone, other steroids, methotrexate, cladribine and cyclophosphamide. These immunosuppressive agents have minimal efficacy in treating MS. Current therapy for rheumatoid arthritis (RA) utilizes agents that non-specifically suppress or modulate immune function such as methotrexate, sulfasalazine, hydroxychloroquine, leuflonamide, prednisone, as well as the recently developed TNFα antagonists etanercept and infliximab (Moreland et al., J Rheumatol, 28:1431-52 (2001)). Etanercept and infliximab globally block TNFα, making patients more susceptible to death from sepsis, aggravation of chronic mycobacterial infections, and development of demyelinating events.

In the case of organ-specific autoimmunity, a number of different therapeutic approaches have been tried. Soluble protein antigens have been administered systemically to inhibit the subsequent immune response to that antigen. Such therapies include delivery of myelin basic protein, its dominant peptide, or a mixture of myelin proteins to animals with experimental autoimmune encephalomyelitis and humans with multiple sclerosis (Brocke et al., Nature, 379:343-6 (1996); Critchfield et al., Science, 263:1139-43 (1994); Weiner et al., Annu Rev Immunol, 12:809-37 (1994)), administration of type II collagen or a mixture of collagen proteins to animals with collagen-induced arthritis and humans with rheumatoid arthritis (Gumanovskaya et al., Immunology, 97:466-73 (1999); McKown et al., Arthritis Rheum, 42:1204-8 (1999); Trentham et al., Science, 261:1727-30 (1993), delivery of insulin to animals and humans with autoimmune diabetes (Pozzilli and Gisella Cavallo, Diabetes Metab Res Rev, 16:306-7 (2000), and delivery of S-antigen to animals and humans with autoimmune uveitis (Nussenblatt et al., Am J Ophthalmol, 123:583-92 (1997). A problem associated with this approach is T cell unresponsiveness induced by systemic injection of antigen. Another approach is the attempt to design rational therapeutic strategies for the systemic administration of a peptide antigen based on the specific interaction between the T cell receptors and peptides bound to MHC molecules. One study using the peptide approach in an animal model of diabetes, resulted in the development of antibody production to the peptide (Hurtenbach, U. et al., J Exp. Med, 177:1499 (1993)). Another approach is the administration of T cell receptor (TCR) peptide immunization. See, e.g., Vandenbark, A. A. et al., Nature, 341:541 (1989). Still another approach is the induction of oral tolerance by ingestion of peptide or protein antigens. See, e.g., Weiner, H. L., Immmunol Today, 18:335 (1997).

Immune responses are currently altered by delivering proteins, polypeptides, or peptides, alone or in combination with adjuvants (immunostimulatory agents). For example, the hepatitis B virus vaccine contains recombinant hepatitis B virus surface antigen, a non-self antigen, formulated in aluminum hydroxide, which serves as an adjuvant. This vaccine induces an immune response against hepatitis B virus surface antigen to protect against infection. An alternative approach involves delivery of an attenuated, replication deficient, and/or non-pathogenic form of a virus or bacterium, each non-self antigens, to elicit a host protective immune response against the pathogen. For example, the oral polio vaccine is composed of a live attenuated virus, a non-self antigen, which infects cells and replicates in the vaccinated individual to induce effective immunity against polio virus, a foreign or non-self antigen, without causing clinical disease. Alternatively, the inactivated polio vaccine contains an inactivated or ‘killed’ virus that is incapable of infecting or replicating and is administered subcutaneously to induce protective immunity against polio virus.

### DNA Vaccination/Polynucleotide Therapy

Polynucleotide therapy, or DNA vaccination, is an efficient method to induce immunity against foreign pathogens (Davis, 1997; Hassett and Whitton, 1996; and Ulmer et al., 1996) and cancer antigens (Stevenson et al., 2004) and to modulate autoimmune processes (Waisman et al., 1996). Following intramuscular injection, plasmid DNA is taken up by, for example, muscle cells allowing for the expression of the encoded polypeptide (Wolff et al., 1992) and the mounting of a long-lived immune response to the expressed proteins (Hassett et al., 2000). In the case of autoimmune disease, the effect is a shift in an ongoing immune response to suppress autoimmune destruction and is believed to include a shift in self-reactive lymphocytes from a Th1- to a Th2-type response. The modulation of the immune response may not be systemic but occur only locally at the target organ under autoimmune attack.

Administration of a polynucleotide encoding a self protein, polypeptide or peptide formulated in precipitation- and/or transfection-facilitating agents or using viral vectors differs from traditional “gene therapy.” Gene therapy is the delivery of a polynucleotide to provide expression of a protein or peptide, to replace a defective or absent protein or peptide in the host and/or to augment a desired physiologic function. Gene therapy includes methods that result in the integration of DNA into the genome of an individual for therapeutic purposes. Examples of gene therapy include the delivery of DNA encoding clotting factors for hemophilia, adenosine deaminase for severe combined immunodeficiency, low-density lipoprotein receptor for familial hypercholesterolemia, glucocerebrosidase for Gaucher's disease, α1-antitrypsin for α1-antitrypsin deficiency, α- or β-globin genes for hemoglobinopathies, and chloride channels for cystic fibrosis (Verma and Somia, Nature, 389:239-42 (1997).

Investigators have described DNA therapies encoding immune molecules to treat autoimmune diseases. Such DNA therapies include DNA encoding the antigen-binding regions of the T cell receptor to alter levels of autoreactive T cells driving the autoimmune response (Waisman et al., Nat Med, 2:899-905 (1996) (U.S. Pat. No. 5,939,400). DNA encoding autoantigens were attached to particles and delivered by gene gun to the skin to prevent multiple sclerosis and collagen induced arthritis. (International Patent Application Publication Nos. WO 97/46253; Ramshaw et al., Immunol. and Cell Bio., 75:409-413 (1997). DNA encoding adhesion molecules, cytokines (e.g., TNFα), chemokines (e.g., C—C chemokines), and other immune molecules (e.g., Fas-ligand) have been used in animal models of autoimmune disease (Youssef et al., J Clin Invest, 106:361-371 (2000); Wildbaum et al., J Clin Invest, 106:671-679 (2000); Wildbaum et al., J Immunol, 165:5860-5866 (2000); Wildbaum et al., J Immunol, 161:6368-7634 (1998); Youssef et al., J Autoimmun, 13:21-9 (1999)). Methods for treating autoimmune disease by administering a nucleic acid encoding one or more autoantigens are described in International Patent Application Nos. WO 00/53019, WO 2003/045316, and WO 2004/047734. While these methods have been successful, further improvements are still needed.

It is an object of the present invention to provide a method of treating or preventing a disease associated with self-protein(s), polypeptide(s), or -peptide(s) that are present and involved in a non-physiological process in an animal. Another object of this invention is to provide a specific method for treating or preventing autoimmune diseases that does not impair the immune system generally. Still another object of the present invention is to provide a specific method for treating or preventing neurodegenerative diseases. Yet another object of the present invention is to provide a composition for treating or preventing a disease associated with self-protein(s), polypeptide(s), or -peptide(s) that is present non-physiologically in an animal. Still another object of this invention is to identify self-protein(s), polypeptide(s), or -peptide(s) that are present non-physiologically and associated with a disease. These and other objects of this invention will be apparent from the specification as a whole.

### BRIEF SUMMARY OF THE INVENTION

The present invention provides novel methods of treating or preventing a disease in an animal associated with one or more self-protein(s), -polypeptide(s), or -peptide(s) that is present in the animal nonphysiologically comprising administering to the animal a self-vector comprising a polynucleotide encoding the self-protein(s), -polypeptide(s) or -peptide(s) associated with the disease. Administration of the self-vector comprising a polynucleotide encoding the self-protein(s), -polypeptide(s) or -peptide(s) modulates an immune response to the self-protein(s), polypeptide(s) or peptide(s) that is expressed by the self-vector. A composition comprising a polynucleotide encoding one or more self-protein(s), -polypeptide(s), or -peptide(s) that is present non-physiologically in a treated animal is useful in treating a disease associated with the self-protein(s), -polypeptide(s), or -peptide(s) present in and/or the target of a non-physiologic process in the animal. It was the discovery of this invention that administration of a polynucleotide encoding a self-protein(s), -polypeptide(s), or -peptide(s) that is present non-physiologically or targeted by a non-physiologic process modulates an immune response to the self-protein(s), -polypeptide(s), or -peptide(s) to treat the disease associated with the self-protein(s), -polypeptide(s), or -peptide(s) involved non-physiologically in the animal.

In one aspect the present invention provides a method of treating an autoimmune disease in a subject associated with one or more self-protein(s), -polypeptide(s) or -peptide(s) present in the subject non-physiologically comprising administering to the subject: a self-vector comprising an immunosuppressive vector backbone and a polynucleotide encoding the self-protein(s), -polypeptide(s) or -peptide(s) associated with the autoimmune disease; and one or more divalent cations at a total concentration greater than physiological levels. In some embodiments, the self-vector backbone is a BHT-1 vector backbone. In some embodiments, the self-vector backbone is non-immunostimulatory (e.g., “immune neutral”).

In some embodiments the one or more divalent cations is selected from the group consisting of Ca²⁺, Mg²⁺, Mn²⁺, Zn²⁺, Al²⁺, Cu²⁺, Ni²⁺, Ba²⁺, Sr²⁺, and mixtures thereof. In some embodiments, the divalent cation is calcium alone. In some embodiment, the divalent cation is a mixture of Ca²⁺ and Mg²⁺.

In some embodiments, the autoimmune disease is multiple sclerosis; in other embodiments, the autoimmune disease is rheumatoid arthritis; and in still other embodiments, the autoimmune disease is lupus. In some embodiments, the self-vector comprises a BHT-1 vector backbone and a polynucleotide encoding human myelin basic protein (MBP); in other embodiments, the self-vector comprises a BHT-1 vector backbone and a polynucleotide encoding human proteolipid protein (PLP); in other embodiments, the self-vector comprises a BHT-1 vector backbone and a polynucleotide encoding human myelin associated glycoprotein (MAG); and in still other embodiments, the self-vector comprises a BHT-1 vector backbone and a polynucleotide encoding human myelin oligodendrocyte protein (MOG). In preferred embodiments, the self-vector is BHT-3009 and is endotoxin-free. In some embodiments, the divalent cation is calcium. In some embodiments, the calcium is at a concentration greater than about 2 mM; in preferred embodiments the calcium is at a concentration of about 5.4 mM.

In another aspect the present invention provides a method of treating multiple sclerosis in a subject comprising administering to the subject a pharmaceutical composition comprising BHT-3009 (SEQ ID NO:3). In some embodiments, the pharmaceutical composition is endotoxin-free. In some embodiments, the pharmaceutical composition further comprises a divalent cation at a concentration greater than physiological levels. In some embodiments, the divalent cation is calcium. In some embodiments, the calcium is at a concentration greater than about 2 mM; in preferred embodiments the calcium is at a concentration of about 5.4 mM.

In another aspect the present invention provides a pharmaceutical composition comprising: a self-vector comprising an immunosuppressive vector backbone and a polynucleotide encoding one or more self-protein(s), -polypeptide(s) or -peptide(s) associated with an autoimmune disease; and a divalent cation at a concentration greater than physiological levels. In some embodiments, the self-vector backbone is a BHT-1 vector backbone. In some embodiments, the self-vector backbone is non-immunostimulatory (e.g., “immune neutral”).

In some embodiments, the autoimmune disease is multiple sclerosis; in other embodiments, the autoimmune disease is rheumatoid arthritis; and in still other embodiments, the autoimmune disease is lupus. In some embodiments, the self-vector of the pharmaceutical composition comprises a BHT-1 vector backbone and a polynucleotide encoding human myelin basic protein (MBP); in other embodiments, the self-vector comprises a BHT-1 vector backbone and a polynucleotide encoding human proteolipid protein (PLP); in other embodiments, the self-vector comprises a BHT-1 vector backbone and a polynucleotide encoding human myelin associated glycoprotein (MAG); and in still other embodiments, the self-vector comprises a BHT-1 vector backbone and a polynucleotide encoding human myelin oligodendrocyte protein (MOG). In preferred embodiments, the self-vector of the pharmaceutical composition is BHT-3009 and is endotoxin-free. In some embodiments, the divalent cation is calcium. In some embodiments, the calcium is at a concentration greater than about 2 mM; in preferred embodiments the calcium is at a concentration of about 5.4 mM.

In another aspect the present invention provides pharmaceutical compositions comprising BHT-3009. The compositions of the invention are typically endotoxin free and may further comprise calcium at a concentration greater than about 2 mM.

### BRIEF DESCRIPTION OF THE DRAWINGS

FIG. 1: Structural Vector Diagram of BHT-3009: The self-vector BHT-3009 is shown with its component parts labeled. A CMV promoter drives expression of human myelin basic protein (MBP). Bovine growth hormone termination and polyA sequences (bGH pA) are encorporated 3′ to hMBP. Vector propogation and selection is accomplished via pUC origin of replication and a Kanamycin resistance gene (Kanr), respectively. BHT-3009 is 3485 basepairs and the location of each component is specified to the left of the vector map.

FIG. 2: Phase I Trial Design: Thirty MS patients were assigned to one of three BHT-3009 dose cohorts. For each dose cohort, patients were randomized into one of the following treatment arms: Arm A: BHT-placebo+atorvastatin-placebo (4 patients); Arm B: BHT-3009+atorvastatin-placebo (3 patients); and Arm C: BHT-3009+atorvastatin (3 patients). Patients randomized to Arm A were re-randomized to open label treatment with one of the following: Arm D: BHT-3009 alone (2 patients) or Arm E: BHT-3009+atorvastatin (2 patients) and were treated and evaluated as patients originally randomized to Arms B or C.

FIG. 3 illustrates improved protein production when transfecting a BHT-1 vector backbone using higher than physiological concentrations of calcium. BHT-3021 (0.25 mg/ml) DNA, a BHT-1 vector backbone with a sequence encoding a proinsulin self-protein, was formulated in Dulbecco's PBS with increasing concentrations of calcium ranging from 0.9 mM-9.0 mM in the absence of magnesium. The formulated DNA was frozen overnight to promote the formation of DNA/Calcium phosphate particles. The solution was then thawed and 5 micrograms of DNA was added to ˜3×10⁵ HEK293 cells in a 24-well tissue culture plate containing 0.4 ml DMEM culture media. After 24 hours of culture the cells were treated with a proteasome inhibitor to prevent the degradation of the cytoplasmic proinsulin protein produced by the plasmid and then following another 24 hours of culture cells were harvested, lysed, and proinsulin protein was measured using a commercial proinsulin ELISA kit. Maximum protein production was observed for DNA formulated with 5.4 mM calcium.

### DETAILED DESCRIPTION OF THE INVENTION

In order that the invention described herein may be more fully understood, the following description is set forth.

The present invention provides a method of treating or preventing a disease in an animal associated with one or more self-protein(s), -polypeptide(s) or -peptide(s) present in the animal non-physiologically or involved in a non-physiologic state comprising administering to the animal a self-vector comprising a polynucleotide encoding the self-protein(s), -polypeptide(s) or -peptide(s) associated with the disease. Administration of the self-vector comprising a polynucleotide encoding the self-protein(s), -polypeptide(s) or -peptide(s) modulates an immune response to the self-protein(s), -polypeptide(s) or -peptide(s) expressed from the self-vector.

The self-vector is co-administered or co-formulated with one or more divalent cations present at higher than physiologic concentrations. Surprisingly, co-administration of a DNA vaccination vector with one or more divalent cations at total concentration higher than physiologic levels improves one or more of transfection efficiency, expression (i.e., transcription and translation) of the encoded autoantigen, and therapeutic suppression of an undesirable immune response in comparison to co-administration of a DNA vaccination vector in the presence of one or more divalent cations at total concentration equal to or lower than physiologic levels.

The method of treatment or prevention of this invention can be used for any disease associated with a self-protein(s), -polypeptide(s) or -peptide(s) that is present non-physiologically and/or involved in a non-physiologic process within the animal.

### Autoimmune Diseases

Several examples of autoimmune diseases associated with self-protein(s), -polypeptide(s) or -peptide(s) present in the animal non-physiologically is set forth in the table below and is described below.

TABLE 2

| | | Self-Protein(s) Associated With An Autoimmune |
|---|---|---|
| Autoimmune Disease | Tissue Targeted | Disease |
| Multiple sclerosis | central nervous | myelin basic protein, proteolipid protein, myelin |
| | system | associated glycoprotein, cyclic nucleotide |
| | | phosphodiesterase, myelin-associated glycoprotein, |
| | | myelin-associated oligodendrocytic basic protein; |
| | | alpha-B-crystalin |
| Guillian Barre | peripheral nerv. sys. | peripheral myelin protein I and others |
| Syndrome | | |
| Insulin Dependent | β cells in islets of | tyrosine phosphatase IA2, IA-2b; glutamic acid |
| Diabetes Mellitus | pancreas | decarboxylase (65 and 67 kDa forms), |
| | | carboxypeptidase H, insulin, proinsulin, pre- |
| | | proinsulin, heat shock proteins, glima 38, isleT cell |
| | | antigen 69 KDa, p52, islet cell glucose transporter |
| | | GLUT-2 |
| Rheumatoid Arthritis | synovial joints | Immunoglobulin, fibrin, filaggrin, type I, II, III, IV, |
| | | V, IX, and XI collagens, GP-39, hnRNPs |
| Autoimmune Uveitis | eye, uvea | S-antigen, interphotoreceptor retinoid binding |
| | | protein (IRBP), rhodopsin, recoverin |
| Primary Biliary | biliary tree of liver | pyruvate dehydrogenase complexes (2-oxoacid |
| Cirrhosis | | dehydrogenase) |
| Autoimmune Hepatitis | Liver | Hepatocyte antigens, cytochrome P450 |
| Pemphigus vulgaris | Skin | Desmoglein-1, -3, and others |
| Myasthenia Gravis | nerve-muscle junct. | acetylcholine receptor |
| Autoimmune gastritis | stomach/parietal cells | H⁺/K⁺ ATPase, intrinsic factor |
| Pernicious Anemia | Stomach | intrinsic factor |
| Polymyositis | Muscle | histidyl tRNA synthetase, other synthetases, other |
| | | nuclear antigens |
| Autoimmune | Thyroid | Thyroglobulin, thyroid peroxidase |
| Thyroiditis | | |
| Graves's Disease | Thyroid | Thyroid-stimulating hormone receptor |
| Psoriasis | Skin | Unknown |
| Vitiligo | Skin | Tyrosinase, tyrosinase-related protein-2 |
| Systemic Lupus Eryth. | Systemic | nuclear antigens: DNA, histones, |
| | | ribonucleoproteins |
| Celiac Disease | Small bowel | Transglutaminase |

Multiple Sclerosis. The present invention provides compositions and methods useful for treating multiple sclerosis (MS), which is the most common demyelinating disorder of the CNS and affects 350,000 Americans and one million people worldwide. See, e.g., Cohen and Rudick (eds. 2007) Multiple Sclerosis Therapeutics (3d ed) Informa Healthcare, ISBN-10: 1841845256, ISBN-13: 978-1841845258; Matthews and Margaret Rice-Oxley (2006) Multiple Sclerosis: The Facts (Oxford Medical Publications 4th Ed.) Oxford University Press, USA, ISBN-10: 0198508980, ISBN-13: 978-0198508984; Cook (ed. 2006) Handbook of Multiple Sclerosis (Neurological Disease and Therapy, 4th Ed.) Informa Healthcare, ISBN-10: 1574448277, ISBN-13: 978-1574448276; Compston, et al. (2005) McAlpine's Multiple Sclerosis (4th edition) Churchill Livingstone, ISBN-10: 044307271X, ISBN-13: 978-0443072710; Burks and Johnson (eds 2000) Multiple Sclerosis: Diagnosis, Medical Management, and Rehabilitation Demos Medical Publishing ISBN-10: 1888799358, ISBN-13: 978-1888799354; Waxman (2005) Multiple Sclerosis As A Neuronal Disease Academic Press ISBN-10: 0127387617, ISBN-13: 978-0127387611; Filippi, et al. (eds.) Magnetic Resonance Spectroscopy in Multiple Sclerosis (Topics in Neuroscience) Springer, ISBN-10: 8847001234, ISBN-13: 978-8847001237; Herndon (ed. 2003) Multiple Sclerosis: Immunology, Pathology and Pathophysiology Demos Medical Publishing, ISBN-10: 1888799625, ISBN-13: 978-1888799620; Costello, et al. (2007) “Combination therapies for multiple sclerosis: scientific rationale, clinical trials, and clinical practice” Curr. Opin. Neurol. 20(3):281-285, PMID: 17495621; Burton and O'Connor (2007) “Novel Oral Agents for Multiple Sclerosis” Curr. Neurol. Neurosci. Rep. 7(3):223-230, PMID: 17488588; Correale and Villa (2007) “The blood-brain-barrier in multiple sclerosis: functional roles and therapeutic targeting” Autoimmunity 40(2):148-60, PMID: 17453713; De Stefano, et al. (2007) “Measuring brain atrophy in multiple sclerosis” J Neuroimaging 17 Suppl 1:1 OS-15S, PMID: 17425728; Neema, et al. (2007) “T1- and T2-based MRI measures of diffuse gray matter and white matter damage in patients with multiple sclerosis” J. Neuroimaging 17 Suppl 1:16S-21S, PMID: 17425729; De Stefano and Filippi (2007) “MR spectroscopy in multiple sclerosis” J. Neuroimaging 17 Suppl 1:31S-35S, PMID: 17425732; and Comabella and Martin (2007) “Genomics in multiple sclerosis-Current state and future directions” J. Neuroimmunol. Epub ahead of print] PMID: 17400297.

Onset of symptoms typically occurs between 20 and 40 years of age and manifests as an acute or sub-acute attack of unilateral visual impairment, muscle weakness, paresthesias, ataxia, vertigo, urinary incontinence, dysarthria, or mental disturbance (in order of decreasing frequency). Such symptoms result from focal lesions of demyelination which cause both negative conduction abnormalities due to slowed axonal conduction, and positive conduction abnormalities due to ectopic impulse generation (e.g., Lhermitte's symptom). Diagnosis of MS is based upon a history including at least two distinct attacks of neurologic dysfunction that are separated in time, produce objective clinical evidence of neurologic dysfunction, and involve separate areas of the CNS white matter. Laboratory studies providing additional objective evidence supporting the diagnosis of MS include magnetic resonance imaging (MRI) of CNS white matter lesions, cerebral spinal fluid (CSF) oligoclonal banding of IgG, and abnormal evoked responses. Although most patients experience a gradually progressive relapsing remitting disease course, the clinical course of MS varies greatly between individuals and can range from being limited to several mild attacks over a lifetime to fulminant chronic progressive disease. A quantitative increase in myelin-autoreactive T cells with the capacity to secrete IFN-gamma is associated with the pathogenesis of MS and EAE.

The self-protein, -polypeptide or -peptide targets of the autoimmune response in autoimmune demyelinating diseases, such as multiple sclerosis and experimental autoimmune encephalomyelitis (EAE), may comprise epitopes from proteolipid protein (PLP); myelin basic protein (MBP); myelin oligodendrocyte protein (MOG); cyclic nucleotide phosphodiesterase (CNPase); myelin-associated glycoprotein (MAG), and myelin-associated oligodendrocytic basic protein (MBOP); alpha-B-crystalin (a heat shock protein); viral and bacterial mimicry peptides, e.g., influenza, herpes viruses, hepatitis B virus, etc.; OSP (oligodendrocyte specific-protein); citrulline-modified MBP (the C8 isoform of MBP in which 6 arginines have been de-imminated to citrulline), etc. The integral membrane protein PLP is a dominant autoantigen of myelin. Determinants of PLP antigenicity have been identified in several mouse strains, and include residues 139-151, 103-116, 215-232, 43-64 and 178-191. At least 26 MBP epitopes have been reported (Meinl et al., J Clin Invest, 92:2633-43 (1993)). Notable are residues 1-11, 59-76 and 87-99. Immunodominant MOG epitopes that have been identified in several mouse strains include residues 1-22, 35-55, 64-96. As used herein the term “epitope” is understood to mean a portion of a self-protein, -polypeptide, or -peptide having a particular shape or structure that is recognized by either B cells or T cells of the animal's immune system.

In human MS patients the following myelin proteins and epitopes were identified as targets of the autoimmune T and B cell response. Antibody eluted from MS brain plaques recognized myelin basic protein (MBP) peptide 83-97 (Wucherpfennig et al., J Clin Invest, 100:1114-1122 (1997)). Another study found approximately 50% of MS patients having peripheral blood lymphocyte (PBL) T cell reactivity against myelin oligodendrocyte glycoprotein (MOG) (6-10% control), 20% reactive against MBP (8-12% control), 8% reactive against PLP (0% control), 0% reactive MAG (0% control). In this study, 7 of 10 MOG reactive patients had T cell proliferative responses focused on one of 3 peptide epitopes, including MOG 1-22, MOG 34-56, MOG 64-96 (Kerlero de Rosbo et al., Eur J Immunol, 27:3059-69 (1997)). T and B cell (brain lesion-eluted Ab) response focused on MBP 87-99 (Oksenberg et al., Nature, 362:68-70 (1993)). In MBP 87-99, the amino acid motif HFFK is a dominant target of both the T and B cell response (Wucherpfennig et al., J Clin Invest, 100:1114-22 (1997)). Another study observed lymphocyte reactivity against myelin-associated oligodendrocytic basic protein (MOBP), including residues MOBP 21-39 and MOBP 37-60 (Holz et al., J Immunol, 164:1103-9 (2000)). Using immunogold conjugates of MOG and MBP peptides to stain MS and control brains both MBP and MOG peptides were recognized by MS plaque-bound Abs (Genain and Hauser, Methods, 10:420-34 (1996)).

Rheumatoid Arthritis Rheumatoid arthritis (RA) is a chronic autoimmune inflammatory synovitis affecting 0.8% of the world population. It is characterized by chronic inflammatory synovitis that causes erosive joint destruction. See, e.g., St. Clair, et al. (2004) Rheumatoid Arthritis Lippincott Williams & Wilkins, ISBN-10: 0781741491, ISBN-13: 978-0781741491; Firestein, et al. (eds. 2006) Rheumatoid Arthritis (2d Ed.) Oxford University Press, USA, ISBN-10: 0198566301, ISBN-13: 978-0198566304; Emery, et al. (2007) “Evidence-based review of biologic markers as indicators of disease progression and remission in rheumatoid arthritis” Rheumatol. Int. [Epub ahead of print] PMID: 17505829; Nigrovic, et al. (2007) “Synovial mast cells: role in acute and chronic arthritis” Immunol. Rev. 217(1):19-37, PMID: 17498049; and Manuel, et al. (2007) “Dendritic cells in autoimmune diseases and neuroinflammatory disorders” Front. Biosci. 12:4315-335, PMID: 17485377. RA is mediated by T cells, B cells and macrophages.

Evidence that T cells play a critical role in RA includes the (1) predominance of CD4⁺ T cells infiltrating the synovium, (2) clinical improvement associated with suppression of T cell function with drugs such as cyclosporine, and (3) the association of RA with certain HLA-DR alleles. The HLA-DR alleles associated with RA contain a similar sequence of amino acids at positions 67-74 in the third hypervariable region of the β chain that are involved in peptide binding and presentation to T cells. RA is mediated by autoreactive T cells that recognize a self-protein, or modified self-protein, present in synovial joints. Self-antigens, -protein(s), -polypeptide(s) or -peptides of this invention also referred to as autoantigens are targeted in RA and comprise epitopes from type II collagen; hnRNP; A2/RA33; Sa; filaggrin; keratin; citrulline; cartilage proteins including gp39; collagens type I, III, IV, V, IX, XI; HSP-65/60; IgM (rheumatoid factor); RNA polymerase; hnRNP-B1; hnRNP-D; cardiolipin; aldolase A; citrulline-modified filaggrin and fibrin. Autoantibodies that recognize filaggrin peptides containing a modified arginine residue (de-imminated to form citrulline) have been identified in the serum of a high proportion of RA patients. Autoreactive T and B cell responses are both directed against the same immunodominant type II collagen (CII) peptide 257-270 in some patients.

Insulin Dependent Diabetes Mellitus Human type I or insulin-dependent diabetes mellitus (IDDM) is characterized by autoimmune destruction of the β cells in the pancreatic islets of Langerhans. The depletion of β cells results in an inability to regulate levels of glucose in the blood. See, e.g., Sperling (ed. 2001) Type 1 Diabetes in Clinical Practice (Contemporary Endocrinology) Humana Press, ISBN-10: 0896039315, ISBN-13: 978-0896039315; Eisenbarth (ed. 2000) Type 1 Diabetes: Molecular, Cellular and Clinical Immunology (Advances in Experimental Medicine and Biology) Springer, ISBN-10: 0306478714, ISBN-13: 978-0306478710; Wong and Wen (2005) “B cells in autoimmune diabetes” Rev. Diabet. Stud. 2(3):121-135, Epub 2005 Nov. 10, PMID: 17491687; Sia (2004) “Autoimmune diabetes: ongoing development of immunological intervention strategies targeted directly against autoreactive T cells” Rev. Diabet. Stud. 1(1):9-17, Epub 2004 May 10, PMID: 17491660; Triplitt (2007) “New technologies and therapies in the management of diabetes” Am. J Manag. Care 13(2 Suppl):S47-54, PMID: 17417933; and Skyler (2007) “Prediction and prevention of type 1 diabetes: progress, problems, and prospects” Clin. Pharmacol. Ther. 81(5):768-71, Epub 2007 Mar. 28, PMID: 17392722.

Overt diabetes occurs when the level of glucose in the blood rises above a specific level, usually about 250 mg/dl. In humans a long presymptomatic period precedes the onset of diabetes. During this period there is a gradual loss of pancreatic beta cell function. The development of disease is implicated by the presence of autoantibodies against insulin, glutamic acid decarboxylase, and the tyrosine phosphatase IA2 (IA2), each an example of a self-protein, -polypeptide or -peptide according to this invention.

Markers that may be evaluated during the presymptomatic stage are the presence of insulitis in the pancreas, the level and frequency of isleT cell antibodies, isleT cell surface antibodies, aberrant expression of Class II MHC molecules on pancreatic beta cells, glucose concentration in the blood, and the plasma concentration of insulin. An increase in the number of T lymphocytes in the pancreas, isleT cell antibodies and blood glucose is indicative of the disease, as is a decrease in insulin concentration.

The Non-Obese Diabetic (NOD) mouse is an animal model with many clinical, immunological, and histopathological features in common with human IDDM. NOD mice spontaneously develop inflammation of the islets and destruction of the β cells, which leads to hyperglycemia and overt diabetes. Both CD4⁺ and CD8⁺ T cells are required for diabetes to develop, although the roles of each remain unclear. It has been shown that administration of insulin or GAD, as proteins, under tolerizing conditions to NOD mice prevents disease and down-regulates responses to the other self-antigens.

The presence of combinations of autoantibodies with various specificities in serum are highly sensitive and specific for human type I diabetes mellitus. For example, the presence of autoantibodies against GAD and/or IA-2 is approximately 98% sensitive and 99% specific for identifying type I diabetes mellitus from control serum. In non-diabetic first degree relatives of type I diabetes patients, the presence of autoantibodies specific for two of the three autoantigens including GAD, insulin and IA-2 conveys a positive predictive value of >90% for development of type I DM within 5 years.

Autoantigens targeted in human insulin dependent diabetes mellitus may include the self-protein(s), -polypeptide(s) or -peptide(s) tyrosine phosphatase IA-2; IA-2β; glutamic acid decarboxylase (GAD) both the 65 kDa and 67 kDa forms; carboxypeptidase H; insulin; proinsulin; heat shock proteins (HSP); glima 38; isleT cell antigen 69 KDa (ICA69); p52; two ganglioside antigens (GT3 and GM2-1); and an isleT cell glucose transporter (GLUT 2).

Human IDDM is currently treated by monitoring blood glucose levels to guide injection, or pump-based delivery, of recombinant insulin. Diet and exercise regimens contribute to achieving adequate blood glucose control.

Autoimmune Uveitis Autoimmune uveitis is an autoimmune disease of the eye that is estimated to affect 400,000 people, with an incidence of 43,000 new cases per year in the U.S. Autoimmune uveitis is currently treated with steroids, immunosuppressive agents such as methotrexate and cyclosporin, intravenous immunoglobulin, and TNFα-antagonists. See, e.g., Pleyer and Mondino (eds. 2004) Uveitis and Immunological Disorders (Essentials in Ophthalmology) Springer, ISBN-10: 3540200452, ISBN-13: 978-3540200451; Vallochi, et al. (2007) “The role of cytokines in the regulation of ocular autoimmune inflammation” Cytokine Growth Factor Rev. 18(1-2):135-141, Epub 2007 Mar. 8, PMID: 17349814; Bora and Kaplan (2007) “Intraocular diseases—anterior uveitis” Chem. Immunol. Allergy. 92:213-20, PMID: 17264497; and Levinson (2007) “Immunogenetics of ocular inflammatory disease” Tissue Antigens 69(2):105-112, PMID: 17257311.

Experimental autoimmune uveitis (EAU) is a T cell-mediated autoimmune disease that targets neural retina, uvea, and related tissues in the eye. EAU shares many clinical and immunological features with human autoimmune uveitis, and is induced by peripheral administration of uveitogenic peptide emulsified in Complete Freund's Adjuvant (CFA).

Self-proteins targeted by the autoimmune response in human autoimmune uveitis may include S-antigen, interphotoreceptor retinoid binding protein (IRBP), rhodopsin, and recovern.

Primary Biliary Cirrhosis Primary Biliary Cirrhosis (PBC) is an organ-specific autoimmune disease that predominantly affects women between 40-60 years of age. The prevalence reported among this group approaches 1 per 1,000. PBC is characterized by progressive destruction of intrahepatic biliary epithelial cells (IBEC) lining the small intrahepatic bile ducts. This leads to obstruction and interference with bile secretion, causing eventual cirrhosis. Association with other autoimmune diseases characterized by epithelium lining/secretory system damage has been reported, including Sjogren's Syndrome, CREST Syndrome, Autoimmune Thyroid Disease and Rheumatoid Arthritis. Attention regarding the driving antigen(s) has focused on the mitochondria for over 50 years, leading to the discovery of the antimitochondrial antibody (AMA) (Gershwin et al., Immunol Rev, 174:210-225 (2000); Mackay et al., Immunol Rev, 174:226-237 (2000)). AMA soon became a cornerstone for laboratory diagnosis of PBC, present in serum of 90-95% patients long before clinical symptoms appear. Autoantigenic reactivities in the mitochondria were designated as M1 and M2. M2 reactivity is directed against a family of components of 48-74 kDa. M2 represents multiple autoantigenic subunits of enzymes of the 2-oxoacid dehydrogenase complex (2-OADC) and is another example of the self-protein, -polypeptide, or -peptide of the instant invention.

Studies identifying the role of pyruvate dehydrogenase complex (PDC) antigens in the etiopathogenesis of PBC support the concept that PDC plays a central role in the induction of the disease (Gershwin et al., Immunol Rev, 174:210-225 (2000); Mackay et al., Immunol Rev, 174:226-237 (2000)). The most frequent reactivity in 95% of cases of PBC is the E2 74 kDa subunit, belonging to the PDC-E2. There exist related but distinct complexes including: 2-oxoglutarate dehydrogenase complex (OGDC) and branched-chain (BC) 2-OADC. Three constituent enzymes (E1, 2, 3) contribute to the catalytic function which is to transform the 2-oxoacid substrate to acyl co-enzyme A (CoA), with reduction of NAD⁺ to NADH. Mammalian PDC contains an additional component, termed protein X or E-3 Binding protein (E3BP). In PBC patients, the major antigenic response is directed against PDC-E2 and E3BP. The E2 polypeptide contains two tandemly repeated lipoyl domains, while E3BP has a single lipoyl domain. PBC is treated with glucocorticoids and immunosuppressive agents including methotrexate and cyclosporin A. See, e.g., Sherlock and Dooley (2002) Diseases of the Liver & Biliary System (11th ed.) Blackwell Pub., ISBN-10: 0632055820, ISBN-13: 978-0632055821; Boyer, et al. (eds. 2001) Liver Cirrhosis and its Development (Falk Symposium, Volume 115) Springer, ISBN-10: 0792387600, ISBN-13: 978-0792387602; Crispe (ed. 2001) T Lymphocytes in the Liver: Immunobiology, Pathology and Host Defense Wiley-Liss, ISBN-10: 047119218X, ISBN-13: 978-0471192183; Lack (2001) Pathology of the Pancreas, Gallbladder, Extrahepatic Biliary Tract, and Ampullary Region (Medicine) Oxford University Press, USA, ISBN-10: 0195133927, ISBN-13: 978-0195133929; Gong, et al. (2007) “Ursodeoxycholic Acid for Patients With Primary Biliary Cirrhosis: An Updated Systematic Review and Meta-Analysis of Randomized Clinical Trials Using Bayesian Approach as Sensitivity Analyses” Am. J. Gastroenterol. [Epub ahead of print] PMID: 17459023; Lazaridis and Talwalkar (2007) “Clinical Epidemiology of Primary Biliary Cirrhosis: Incidence, Prevalence, and Impact of Therapy” J. Clin. Gastroenterol. 41(5):494-500, PMID: 17450033; and Sorokin, et al. (2007) “Primary biliary cirrhosis, hyperlipidemia, and atherosclerotic risk: A systematic review” Atherosclerosis [Epub ahead of print] PMID: 17240380.

A murine model of experimental autoimmune cholangitis (EAC) uses intraperitoneal (i.p.) sensitization with mammalian PDC in female SJL/J mice, inducing non-suppurative destructive cholangitis (NSDC) and production of AMA (Jones, J Clin Pathol, 53:813-21 (2000)).

Other Autoimmune Diseases And Associated Self-Protein(s), -polypeptide(s) Or -Peptide(s). Autoantigens for myasthenia gravis may include epitopes within the acetylcholine receptor. Autoantigens targeted in pemphigus vulgaris may include desmoglein-3. Sjogren's syndrome antigens may include SSA (Ro); SSB (La); and fodrin. The dominant autoantigen for pemphigus vulgaris may include desmoglein-3. Panels for myositis may include tRNA synthetases (e.g., threonyl, histidyl, alanyl, isoleucyl, and glycyl); Ku; Scl; SSA; U1 Sn ribonuclear protein; Mi-1; Mi-1; Jo-1; Ku; and SRP. Panels for scleroderma may include Sc1-70; centromere; U1 ribonuclear proteins; and fibrillarin. Panels for pernicious anemia may include intrinsic factor; and glycoprotein beta subunit of gastric H/K ATPase. Epitope Antigens for systemic lupus erythematosus (SLE) may include DNA; phospholipids; nuclear antigens; Ro; La; U1 ribonucleoprotein; Ro60 (SS-A); Ro52 (SS-A); La (SS-B); calreticulin; Grp78; Sc1-70; histone; Sm protein; and chromatin, etc. For Grave's disease epitopes may include the Na⁺/I⁻ symporter; thyrotropin receptor; Tg; and TPO.

### Polynucleotide Therapy—Materials and Methods

Before describing the present invention in detail, it is to be understood that this invention is not limited to particular formulations or process parameters as they may, of course, vary. It is also to be understood that the terminology used herein is for the purpose of describing particular embodiments of the invention only, and is not intended to be limiting.

Although a number of materials and methods similar or equivalent to those described herein can be used in the practice of the present invention, the preferred materials and methods are described herein.

The terms “polynucleotide” and “nucleic acid” refer to a polymer composed of a multiplicity of nucleotide units (ribonucleotide or deoxyribonucleotide or related structural variants) linked via phosphodiester bonds. A polynucleotide or nucleic acid can be of substantially any length, typically from about six (6) nucleotides to about 109 nucleotides to about 4000 nucleotides or larger. Polynucleotides and nucleic acids include RNA, DNA, synthetic forms, and mixed polymers, both sense and antisense strands, double- or single-stranded, and can also be chemically or biochemically modified or can contain non-natural or derivatized nucleotide bases, as will be readily appreciated by the skilled artisan. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The term “promoter” is used here to refer to the polynucleotide region recognized by RNA polymerases for the initiation of RNA synthesis, or “transcription”. Promoters are one of the functional elements of self-vectors that regulate the efficiency of transcription and thus the level of protein expression of a self-polypeptide encoded by a self-vector. Promoters can be “constitutive”, allowing for continual transcription of the associated gene, or “inducible”, and thus regulated by the presence or absence of different substances in the environment. Additionally, promoters can also either be general, for expression in a broad range of different cell types, or cell-type specific, and thus only active or inducible in a particular cell type, such as a muscle cell. Promoters controlling transcription from vectors may be obtained from various sources, for example, the genomes of viruses such as: polyoma, simian virus 40 (SV40), adenovirus, retroviruses, hepatitis B virus and preferably cytomegalovirus, or from heterologous mammalian promoters, e.g., b-actin promoter. The early and late promoters of the SV40 virus are conveniently obtained as is the immediate early promoter of the human cytomegalovirus.

“Enhancer” refers to cis-acting polynucleotide regions of about from 10-300 basepairs that act on a promoter to enhance transcription from that promoter. Enhancers are relatively orientation and position independent and can be placed 5′ or 3′ to the transcription unit, within introns, or within the coding sequence itself.

A “terminator sequence” as used herein means a polynucleotide sequence that signals the end of DNA transcription to the RNA polymerase. Often the 3′ end of a RNA generated by the terminator sequence is then processed considerably upstream by polyadenylation. “Polyadenylation” is used to refer to the non-templated addition of about 50 to about 200 nucleotide chain of polyadenylic acid (polyA) to the 3′ end of a transcribed messenger RNA. The “polyadenylation signal” (AAUAAA) is found within the 3′ untranslated region (UTR) of a mRNA and specifies the site for cleavage of the transcript and addition of the polyA tail. Transcription termination and polyadenylation are functionally linked and sequences required for efficient cleavage/polyadenylation also constitute important elements of termination sequences (Connelly and Manley, 1988).

The terms “DNA vaccination”, “DNA immunization”, and “polynucleotide therapy” are used interchangeably herein and refer to the administration of a polynucleotide to a subject for the purpose of modulating an immune response. “DNA vaccination” with plasmids expressing foreign microbial antigens is a well known method to induce protective antiviral or antibacterial immunity (Davis, 1997; Hassett and Whitton, 1996; and Ulmer et al., 1996). For the purpose of the present invention, “DNA vaccination”, “DNA immunization”, or “polynucleotide therapy” refers to the administration of polynucleotides encoding one or more self-polypeptides that include one or more autoantigenic epitopes associated with a disease. The “DNA vaccination” serves the purpose of modulating an ongoing immune response to suppress autoimmune destruction for the treatment or prevention of an autoimmune disease. Modulation of an immune response in reaction to “DNA vaccination” may include shifting self-reactive lymphocytes from a Th1- to a Th2-type response. The modulation of the immune response may occur systemically or only locally at the target organ under autoimmune attack.

“Self-vector” means one or more vector(s) which taken together comprise a polynucleotide either DNA or RNA encoding one or more self-protein(s), -polypeptide(s), -peptide(s). Polynucleotide, as used herein is a series of either deoxyribonucleic acids including DNA or ribonucleic acids including RNA, and their derivatives, encoding a self-protein, -polypeptide, or -peptide of this invention. The self-protein, -polypeptide or -peptide coding sequence is inserted into an appropriate plasmid expression self-cassette. Once the polynucleotide encoding the self-protein, -polypeptide, or -peptide is inserted into the expression self-cassette the vector is then referred to as a “self-vector.” In the case where polynucleotide encoding more than one self-protein(s), -polypeptide(s), or -peptide(s) is to be administered, a single self-vector may encode multiple separate self-protein(s), -polypeptide(s) or -peptide(s). In one embodiment, DNA encoding several self-protein(s), -polypeptide(s), or -peptide(s) are encoded sequentially in a single self-plasmid utilizing internal ribosomal re-entry sequences (IRES) or other methods to express multiple proteins from a single DNA molecule. The DNA expression self-vectors encoding the self-protein(s), -polypeptide(s), or -peptide(s) are prepared and isolated using commonly available techniques for isolation of plasmid DNA such as those commercially available from Qiagen Corporation. The DNA is purified free of bacterial endotoxin for delivery to humans as a therapeutic agent. Alternatively, each self-protein, -polypeptide or -peptide is encoded on a separate DNA expression vector.

The term “vector backbone” refers to the portion of a plasmid vector other than the sequence encoding a self-antigen, -protein, -polypeptide, or -peptide.

An “immunosuppressive vector backbone” refers to a vector backbone that either (i) elicits a reduced immune response in comparison to a parent vector backbone, or (ii) prevents or inhibits an immune response. The immune response can be measured using in vitro or in vivo assays known in the art. For example, the immune response can be determined by measuring proliferation of lymphocytes exposed to the vector backbone, or by measuring production of cytokines (in cell culture media, in serum, etc.) indicative of immune stimulation (e.g., IL-2, IFN-γ, IL-6). In some embodiments, an immunosuppressive vector backbone contains fewer immunostimulatory sequences (e.g., CpG sequences) in comparison to a parent vector backbone. In some embodiments, an immunosuppressive vector backbone contains one or more immunoinhibitory sequences (IIS), for example, as described herein and known in the art. In some embodiments, an immunosuppressive vector backbone promotes a Th2 immune response and inhibits a Th1 immune response.

“Self-antigen, -protein, -polypeptide, or -peptide” as used herein refers to any protein, polypeptide, or peptide, or fragment or derivative thereof that: is encoded within the genome of the animal; is produced or generated in the animal; may be modified post-translationally at some time during the life of the animal; and, is present in the animal non-physiologically. Self-antigens, -protein(s), -polypeptide(s) or -peptides of this invention are also referred to as autoantigens. Fragments and derivatives may be generated by deletion of part of the coding sequence, and in certain cases inserting a new ATG start codon encoding a methionine, inserting a new stop codon, and/or deleting, removing or modifying other sequences to generate fragments or derivatives of the self protein, -polypeptide, or -peptide. The term “non-physiological” or “non-physiologically” when used to describe the self-proteins, -polypeptides, or -peptides of this invention means a departure or deviation from the normal role or process in the animal for that self-protein, -polypeptide or -peptide. When referring to the self-protein, -polypeptide or -peptide as “associated with a disease” or “involved in a disease” it is understood to mean that the self-protein, -polypeptide, or -peptide may be modified in form or structure and thus be unable to perform its physiological role or process; or may be involved in the pathophysiology of the condition or disease either by inducing the pathophysiology, mediating or facilitating a pathophysiologic process; and/or by being the target of a pathophysiologic process. For example, in autoimmune disease, the immune system aberrantly attacks self-proteins causing damage and dysfunction of cells and tissues in which the self-protein is expressed and/or present. Examples of posttranslational modifications of self-protein(s), polypeptide(s) or -peptide(s) are glycosylation, addition of lipid groups, dephosphorylation by phosphatases, addition of dimethylarginine residues, citrullination of fillagrin and fibrin by peptidyl arginine deiminase (PAD); alpha □ crystallin phosphorylation; citrullination of MBP; and SLE autoantigen proteolysis by caspases and granzymes). Immunologically, self-protein, -polypeptide or -peptide would all be considered host self-antigens and under normal physiological conditions are ignored by the host immune system through the elimination, inactivation, or lack of activation of immune cells that have the capacity to recognize self-antigens through a process designated “immune tolerance.” Antigen refers to a molecule that can be recognized by the immune system that is by B cells or T cells, or both. Self-protein, -polypeptide, or -peptide does not include immune proteins, polypeptides, or peptides which are molecules expressed physiologically, specifically and exclusively by cells of the immune system for the purpose of regulating immune function. The immune system is the defense mechanism that provides the means to make rapid, highly specific, and protective responses against the myriad of potentially pathogenic microorganisms inhabiting the animal's world. Examples of immune protein(s), polypeptide(s) or peptide(s) are proteins comprising the T cell receptor, immunoglobulins, cytokines including the type I interleukins, and the type II cytokines, including the interferons and IL-10, TNF, lymphotoxin, and the chemokines such as macrophage inflammatory protein -1 alpha and beta, monocyte-chemotactic protein and RANTES, and other molecules directly involved in immune function such as Fas-ligand. There are certain immune proteins, polypeptide(s) or peptide(s) that are included in the self-protein, -polypeptide or -peptide of the invention and they are: class I MHC membrane glycoproteins, class II MHC glycoproteins and osteopontin. Self-protein, -polypeptide or -peptide does not include proteins, polypeptides, and peptides that are absent from the subject, either entirely or substantially, due to a genetic or acquired deficiency causing a metabolic or functional disorder, and are replaced either by administration of said protein, polypeptide, or peptide or by administration of a polynucleotide encoding said protein, polypeptide or peptide (gene therapy). Self-protein, -polypeptide or -peptide does not include proteins, polypeptides, and peptides expressed specifically and exclusively by cells which have characteristics that distinguish them from their normal counterparts, including: (1) clonality, representing proliferation of a single cell with a genetic alteration to form a clone of malignant T cells, (2) autonomy, indicating that growth is not properly regulated, and (3) anaplasia, or the lack of normal coordinated cell differentiation. Cells have one or more of the foregoing three criteria are referred to either as neoplastic, cancer or malignant T cells.

“Modulation of, modulating or altering an immune response” as used herein refers to an alteration of existing or potential immune response(s) against self-molecules, including but not limited to nucleic acids, lipids, phospholipids, carbohydrates, self-protein(s), -polypeptide(s), -peptide(s), protein complexes, ribonucleoprotein complexes, or derivative(s) thereof that occurs as a result of administration of a polynucleotide encoding a self-protein, -polypeptide, -peptide, nucleic acid, or a fragment or derivative thereof. Such modulation includes an alteration in presence, capacity or function of an immune cell involved in or capable of being involved in an immune response. Immune cells include B cells, T cells, NK cells, NK T cells, professional antigen-presenting cells, non-professional antigen-presenting cells, inflammatory cells, or another cell capable of being involved in or influencing an immune response. Modulation includes a change imparted on an existing immune response, a developing immune response, a potential immune response, or the capacity to induce, regulate, influence, or respond to an immune response. Modulation includes an alteration in the expression and/or function of genes, proteins and/or other molecules in immune cells as part of an immune response.

Modulation of an immune response includes, but is not limited to: elimination, deletion, or sequestration of immune cells; induction or generation of immune cells that can modulate the functional capacity of other cells such as autoreactive lymphocytes, APCs, or inflammatory cells; induction of an unresponsive state in immune cells, termed anergy; increasing, decreasing or changing the activity or function of immune cells or the capacity to do so, including but not limited to altering the pattern of proteins expressed by these cells. Examples include altered production and/or secretion of certain classes of molecules such as cytokines, chemokines, growth factors, transcription factors, kinases, costimulatory molecules, or other cell surface receptors; or a combination of these modulatory events.

For example, polynucleotides encoding self-protein(s), -polypeptide(s), -peptide(s) can modulate immune responses by eliminating, sequestering, or turning-off immune cells mediating or capable of mediating an undesired immune response; inducing, generating, or turning on immune cells that mediate or are capable of mediating a protective immune response; changing the physical or functional properties of immune cells; or a combination of these effects. Examples of measurements of the modulation of an immune response include, but are not limited to, examination of the presence or absence of immune cell populations (using flow cytometry, immunohistochemistry, histology, electron microscopy, the polymerase chain reaction); measurement of the functional capacity of immune cells including ability or resistance to proliferate or divide in response to a signal (such as using T cell proliferation assays and pepscan analysis based on ³H-thymidine incorporation following stimulation with anti-CD3 antibody, anti-T cell receptor antibody, anti-CD28 antibody, calcium ionophores, PMA, antigen presenting cells loaded with a peptide or protein antigen; B cell proliferation assays); measurement of the ability to kill or lyse other cells (such as cytotoxic T cell assays); measurements of the cytokines, chemokines, cell surface molecules, antibodies and other products of the cells (by flow cytometry, enzyme-linked immunosorbent assays, Western blot analysis, protein microarray analysis, immunoprecipitation analysis); measurement of biochemical markers of activation of immune cells or signaling pathways within immune cells (Western blot and immunoprecipitation analysis of tyrosine, serine or threonine phosphorylation, polypeptide cleavage, and formation or dissociation of protein complexes; protein array analysis; DNA transcriptional profiling using DNA arrays or subtractive hybridization); measurements of cell death by apoptosis, necrosis, or other mechanisms (annexin V staining, TUNEL assays, gel electrophoresis to measure DNA laddering, histology; fluorogenic caspase assays, Western blot analysis of caspase substrates); measurement of the genes, proteins, and other molecules produced by immune cells (Northern blot analysis, polymerase chain reaction, DNA microarrays, protein microarrays, 2-dimentional gel electrophoresis, Western blot analysis, enzyme linked immunosorbent assays, flow cytometry); and measurement of clinical outcomes such as improvement of autoimmune, neurodegenerative, and other diseases involving non-physiologic self proteins (clinical scores, requirements for use of additional therapies, functional status, imaging studies).

“Immune Modulatory Sequences (IMSs)” as used herein refers to compounds consisting of deoxynucleotides, ribonucleotides, or analogs thereof that modulate an autoimmune or inflammatory disease. IMSs may be oligonucleotides or a sequence of nucleotides incorporated in a vector. “Oligonucleotide” means multiple nucleotides. Nucleotides are molecules comprising a sugar (preferably ribose or deoxyribose) linked to a phosphate group and an exchangeable organic base, which can be either a substituted purine (guanine (G), adenine (A), or inosine (I)) or a substituted pyrimidine (thymine (T), cytosine (C), or uracil (U)). Oligonucleotide refers to both oligoribonucleotides and to oligodeoxyribonucleotides, herein after referred to as ODNs. ODNs include oligonucleosides (i.e. a oligonucleotide minus the phosphate) and other organic base containing polymers. Oligonucleotide encompasses any length of multiple nucleotides, from a chain of two or more linked nucleotides, and includes chromosomal material containing millions of linked nucleotides.

In certain variations, the method for treating an autoimmune disease includes the administration of an adjuvant for modulating the immune response comprising a CpG oligonucleotide in order to enhance the immune response. CpG oligonucleotides or stimulatory IMSs have been shown to enhance the antibody response of DNA vaccinations (Krieg et al., Nature, 374:546-9 (1995)). The CpG oligonucleotides will consist of a purified oligonucleotide of a backbone that is resistant to degradation in vivo such as a phosphorothioated backbone. The stimulatory IMS useful in accordance with the present invention comprise the following core hexamer:

5′-purine-pyrimidine-[C]-[G]-pyrimidine-pyrimidine-3′

or

5′-purine-purine-[C]-[G]-pyrimidine-pyrimidine-3′;

The core hexamer of immune stimulatory IMSs can be flanked 5′ and/or 3′ by any composition or number of nucleotides or nucleosides. Preferably, stimulatory IMSs range between 6 and 100 base pairs in length, and most preferably 16-50 base pairs in length. Stimulatory IMSs can also be delivered as part of larger pieces of DNA, ranging from 100 to 100,000 base pairs. Stimulatory IMSs can be incorporated in, or already occur in, DNA plasmids, viral vectors and genomic DNA. Most preferably stimulatory IMSs can also range from 6 (no flanking sequences) to 10,000 base pairs, or larger, in size. Sequences present which flank the hexamer core can be constructed to substantially match flanking sequences present in any known immunostimulatory sequences (ISS). For example, the flanking sequences TGACTGTG-Pu-Pu-C-G-Pyr-Pyr-AGAGATGA, where TGACTGTG and AGAGATGA are flanking sequences. Another preferred flanking sequence incorporates a series of pyrimidines (C, T, and U), either as an individual pyrimidine repeated two or more times, or a mixture of different pyrimidines two or more in length. Different flanking sequences have been used in testing inhibitory modulatory sequences and can be adapted to stimulatory modulatory sequences. Further examples of flanking sequences are contained in the following references: U.S. Pat. Nos. 6,225,292 and 6,339,068; and Zeuner et al., Arthritis and Rheumatism, 46:2219-24 (2002).

Particular stimulatory IMSs suitable for administration with modified self-vectors of the invention include oligonucleotides containing the following hexamer sequences:
5′-purine-pyrimidine-[X]-[Y]-pyrimidine-pyrimidine-3′ IMSs containing CG dinucleotide cores: GTCGTT, ATCGTT, GCCGTT, ACCGTT, GTCGCT, ATCGCT, GCCGCT, ACCGCT, GTCGTC, ATCGTC, GCCGTC, ACCGTC, and so forth;

Guanine and inosine can generally substitute for adenine and/or uridine can generally substitute for cytosine or thymine and those substitutions can be made as set forth based on the guidelines above. Alternatively ISS-ODNs can be included into self-vectors as described in detail for IMSs above. A particularly useful ISS includes the mouse optimal CpG element AACGTT. A single ISS or multiple ISSs can be added to a modified self-vector at a single or at multiple sites in the vector as long as other functional electors are not disrupted. In one exemplary example the ISS added to a modified self-vector include a cluster of five mouse optimal CpG elements (AACGTT) immediately upstream of the promoter.

In certain variations, the method for treating autoimmune disease further includes the administration of a polynucleotide comprising an inhibitory IMS or an immune inhibitory sequence (IIS). The IISs useful in accordance with the present invention comprise the following core hexamer:

5′-purine-pyrimidine-[X]-[Y]-pyrimidine-pyrimidine-3′

or

5′-purine-purine-[X]-[Y]-pyrimidine-pyrimidine-3′;

wherein X and Y are any naturally occurring or synthetic nucleotide, except that X and Y cannot be cytosine-guanine.

The core hexamer of IMSs can be flanked 5′ and/or 3′ by any composition or number of nucleotides or nucleosides. Preferably, IMSs range between 6 and 100 base pairs in length, and most preferably 16-50 base pairs in length. IMSs can also be delivered as part of larger pieces of DNA, ranging from 100 to 100,000 base pairs. IMSs can be incorporated in, or already occur in, DNA plasmids, viral vectors and genomic DNA. Most preferably IMSs can also range from 6 (no flanking sequences) to 10,000 base pairs, or larger, in size. Sequences present which flank the hexamer core can be constructed to substantially match flanking sequences present in any known immunoinhibitory sequences (IIS). For example, the flanking sequences TTGACTGTG -Pu-Pyr-X-Y-Pyr-Pyr-AGAGATGA, where TTGACTGTG and AGAGATGA are flanking sequences. Another preferred flanking sequence incorporates a series of pyrimidines (C, T, and U), either as an individual pyrimidine repeated two or more times, or a mixture of different pyrimidines two or more in length. Different flanking sequences have been used in testing inhibitory modulatory sequences. Further examples of flanking sequences for inhibitory oligonucleotides are contained in the following references: U.S. Pat. Nos. 6,225,292 and 6,339,068; and Zeuner et al., Arthritis and Rheumatism, 46:2219-24 (2002).

Particular IISs of the invention include oligonucleotides containing the following hexamer sequences:
1. 5′-purine-pyrimidine-[X]-[Y]-pyrimidine-pyrimidine-3′ IMSs containing GG dinucleotide cores: GTGGTT, ATGGTT, GCGGTT, ACGGTT, GTGGCT, ATGGCT, GCGGCT, ACGGCT, GTGGTC, ATGGTC, GCGGTC, ACGGTC, and so forth.
2. 5′-purine-pyrimidine-[X]-[Y]-pyrimidine-pyrimidine-3′ IMSs containing GC dinucleotides cores: GTGCTT, ATGCTT, GCGCTT, ACGCTT, GTGCCT, ATGCCT, GCGCCT, ACGCCT, GTGCTC, ATGCTC, GCGCTC, ACGCTC, and so forth.

Guanine and inosine substitutes for adenine and/or uridine substitutes for cytosine or thymine and those substitutions can be made as set forth based on the guidelines above.

In certain embodiments of the present invention, the core hexamer region of the IMS is flanked at either the 5′ or 3′ end, or at both the 5′ and 3′ ends, by a polyG region. A “polyG region” or “polyG motif” as used herein means a nucleic acid region consisting of at least two (2) contiguous guanine bases, typically from 2 to 30 or from 2 to 20 contiguous guanines. In some embodiments, the polyG region has from 2 to 10, from 4 to 10, or from 4 to 8 contiguous guanine bases. In certain preferred embodiments, the flanking polyG region is adjacent to the core hexamer. In yet other embodiments, the polyG region is linked to the core hexamer by a non-polyG region (non-polyG linker); typically, the non-polyG linker region has no more than 6, more typically no more than 4 nucleotides, and most typically no more than 2 nucleotides.

In other embodiments of the present invention, the method of treating an autoimmune disease includes the administration of improved immune modulatory sequences comprising:

1.) a hexameric sequence 5′-Purine-Pyrimidine[1]-[X]-[Y]-Pyrimidine[2]-Pyrimidine[3]-3′; wherein X and Y are any naturally occurring or synthetic nucleotide, except that
a. X and Y cannot be cytosine-guanine;
b. X and Y cannot be cytosine-cytosine when Pyrimidine[2] is thymine
c. X and Y cannot be cytosine-thymine when Pyrimidine[1] is cytosine

2.) a CC dinucleotide 5′ to the hexameric sequence wherein the CC dinucleotide is between one to five nucleotides 5′ of the hexameric sequence; and

3.) a polyG region 3′ of the hexameric sequence wherein the polyG comprises at least three contiguous Gs and is between two to five nucleotides 3′ of the hexameric sequence wherein the immune modulatory sequence does not contain cytosine-guanine sequences.

In still other embodiments of the present invention, the method of treating an autoimmune disease includes the administration of improved immune modulatory sequences comprising:

1.) a hexameric sequence 5′-Purine-Pyrimidine-[Y]-[Z]-Pyrimidine-Pyrmidine-3′; wherein X and Y are guanine-guanine;

2.) a CC dinucleotide 5′ to the hexameric sequence wherein the CC dinucleotide is between one to five nucleotides 5′ of the hexameric sequence; and

3.) a polyG region 3′ of the hexameric sequence wherein the polyG comprises between two and ten contiguous Gs and is between two to ten nucleotides 3′ of the hexameric sequence

wherein the immune modulatory sequence does not contain cytosine-guanine sequences.

In preferred embodiments, X and Y of the hexameric sequence are GpG. In other preferred embodiments the hexameric sequence is 5′-GTGGTT-3′. In other preferred embodiments the CC di-nucleotide is two nucleotides 5′ of the hexameric sequence. In other preferred embodiments the polyG region comprises three contiguous guanine bases and is two nucleotides 3′ from the hexameric sequence. In one preferred embodiment the improved immune modulatory sequence is 5′-CCATGTGGTTATGGGT-3′.

IMSs also include suppressive oligonucleotides of at least eight nucleotides in length, wherein the oligonucleotide forms a G-tetrad with a circular dichroism (CD) value of greater than about 2.9 and the number of guanosines is at least two (International Patent Application No. WO 2004/012669, which is incorporated by reference herein in its entirety). CD is defined as the differential absorption of left and right hand circularly polarized light. G-tetrads are G-rich DNA segments that allow complex secondary and/or tertiary structures. More specifically a G-tetrad 1) involves the planar association of four guanosines in a cyclic hydrogen bonding arrangement involving non-Watson Crick base-pairing and 2) requires two of more contiguous guanosines or a hexameric region in which over 50% of the bases are guanosines. Examples include an oligonucleotide with at least one and preferably between two and twenty TTAGGG motifs. Other useful suppressive oligonucleotides include but are not limited to those that conform to one of the following: (TGGGCGGT)x where x is preferably between 2 and 100 and more preferably between 2 and 20; GGGTGGGTGGGTATTACCATTA; TTAGGGTTAGGGTCAACCTTCA; or (G)GG(C/G)AAGCTGGACCTTGGGGG(G)

Oligonucleotides can be obtained from existing nucleic acid sources, including genomic DNA, plasmid DNA, viral DNA and cDNA, but are preferably synthetic oligonucleotides produced by oligonucleotide synthesis. IMS can be part of single-strand or double-stranded DNA, RNA and/or oligonucleosides.

IMSs are preferentially oligonucleotides that contain unmethylated GpG oligonucleotides. Alternative embodiments include IMSs in which one or more adenine or cytosine residues are methylated. In eukaryotic cells, typically cytosine and adenine residues can be methylated.

Oligonucleosides can be incorporated into the internal region and/or 5′ and/or 3′ ends of IMSs, and such oligonucleosides can be used as attachment points for additional self-molecules, including self-lipids, self-protein(s), self-peptide(s), self-polypeptide(s), self-glycolipid(s), self-carbohydrate(s), self-glycoprotein(s), and post-translationally-modified self- protein(s), peptide(s), polypeptide(s), or glycoprotein(s), or as attachment points for additional immune modulatory therapeutics. The termini, phosphate groups, base(s), and sugar moieties can be modified to construct IMSs with additional properties.

IMSs can be stabilized and/or unstabilized oligonucleotides. Stabilized oligonucleotides mean oligonucleotides that are relatively resistant to in vivo degradation by exonucleases, endonucleases and other degradation pathways. Preferred stabilized oligonucleotides have modified phophate backbones, and most preferred oligonucleotides have phophorothioate modified phosphate backbones in which at least one of the phosphate oxygens is replaced by sulfur. Backbone phosphate group modifications, including methylphosphonate, phosphorothioate, phophoroamidate and phosphorodithionate internucleotide linkages, can provide antimicrobial properties on IMSs. The IMSs are preferably stabilized oligonucleotides, preferentially using phosphorothioate stabilized oligonucleotides.

Alternative stabilized oligonucleotides include: alkylphosphotriesters and phosphodiesters, in which the charged oxygen is alkylated; arylphosphonates and alkylphosphonates, which are nonionic DNA analogs in which the charged phosphonate oxygen is replaced by an aryl or alkyl group; or/and oligonucleotides containing hexaethyleneglycol or tetraethyleneglycol, or another diol, at either or both termini. Alternative steric configurations can be used to attach sugar moieties to nucleoside bases in IMSs.

The nucleotide bases of the IMS which flank the competing dinucleotides may be the known naturally occurring bases or synthetic non-natural bases. Oligonucleosides may be incorporated into the internal region and/or termini of the IMS-ON using conventional techniques for use as attachment points for other compounds, including self-lipids, self-protein(s), self-peptide(s), self-polypeptide(s), self-glycolipid(s), self-carbohydrate(s), self-glycoprotein(s), and post-translationally-modified self- protein(s), peptide(s), polypeptide(s), or glycoprotein(s), or as attachment points for additional immune modulatory therapeutics. The base(s), sugar moiety, phosphate groups and termini of the IMS-ON may also be modified in any manner known to those of ordinary skill in the art to construct an IMS-ON having properties desired in addition to the modulatory activity of the IMS-ON. For example, sugar moieties may be attached to nucleotide bases of IMS-ON in any steric configuration.

The techniques for making these phosphate group modifications to oligonucleotides are known in the art and do not require detailed explanation. For review of one such useful technique, the intermediate phosphate triester for the target oligonucleotide product is prepared and oxidized to the naturally occurring phosphate triester with aqueous iodine or with other agents, such as anhydrous amines. The resulting oligonucleotide phosphoramidates can be treated with sulfur to yield phophorothioates. The same general technique (excepting the sulfur treatment step) can be applied to yield methylphosphoamidites from methylphosphonates. For more details concerning phosphate group modification techniques, those of ordinary skill in the art may wish to consult U.S. Pat. Nos. 4,425,732; 4,458,066; 5,218,103 and 5,453,496, as well as Tetrahedron Lett. at 21:4149 25 (1995), 7:5575 (1986), 25:1437 (1984) and Journal Am. ChemSoc., 93:6657 (1987), the disclosures of which are incorporated herein for the purpose of illustrating the level of knowledge in the art concerning the composition and preparation of IMSs.

A particularly useful phosphate group modification is the conversion to the phosphorothioate or phosphorodithioate forms of the IMS-ON oligonucleotides. Phosphorothioates and phosphorodithioates are more resistant to degradation in vivo than their unmodified oligonucleotide counterparts, making the IMS-ON of the invention more available to the host.

IMS-ON can be synthesized using techniques and nucleic acid synthesis equipment which are well-known in the art. For reference in this regard, see, e.g., Ausubel et al., Current Protocols in Molecular Biology, Chs. 2 and 4 (Wiley Interscience, 1989); Maniatis et al., Molecular Cloning: A Laboratory Manual (Cold Spring Harbor Lab., New York, 1982); U.S. Pat. No. 4,458,066 and U.S. Pat. No. 4,650,675. These references are incorporated herein by reference for the purpose of demonstrating the level of knowledge in the art concerning production of synthetic oligonucleotides.

Alternatively, IMS-ON can be obtained by mutation of isolated microbial immune stimulatory sequence (ISS) to substitute a competing dinucleotide for the naturally occurring CpG motif and the flanking nucleotides. Screening procedures which rely on nucleic acid hybridization make it possible to isolate a polynucleotide sequence from any organism, provided the appropriate probe or antibody is available. Oligonucleotide probes, which correspond to a part of the sequence encoding the protein in question, can be synthesized chemically. This requires that short, oligopeptide stretches of amino acid sequence must be known. The DNA sequence encoding the protein can also be deduced from the genetic code, however, the degeneracy of the code must be taken into account.

For example, a cDNA library believed to contain an ISS-containing polynucleotide can be screened by injecting various mRNA derived from cDNAs into oocytes, allowing sufficient time for expression of the cDNA gene products to occur, and testing for the presence of the desired cDNA expression product, for example, by using antibody specific for a peptide encoded by the polynucleotide of interest or by using probes for the repeat motifs and a tissue expression pattern characteristic of a peptide encoded by the polynucleotide of interest. Alternatively, a cDNA library can be screened indirectly for expression of peptides of interest having at least one epitope using antibodies specific for the peptides. Such antibodies can be either polyclonally or monoclonally derived and used to detect expression product indicative of the presence of cDNA of interest.

Once the ISS-containing polynucleotide has been obtained, it can be shortened to the desired length by, for example, enzymatic digestion using conventional techniques. The CpG motif in the ISS-ODN oligonucleotide product is then mutated to substitute an “inhibiting” dinucleotide—identified using the methods of this invention—for the CpG motif. Techniques for making substitution mutations at particular sites in DNA having a known sequence are well known, for example M13 primer mutagenesis through PCR. Because the IMS is non-coding, there is no concern about maintaining an open reading frame in making the substitution mutation. However, for in vivo use, the polynucleotide starting material, ISS-ODN oligonucleotide intermediate or IMS mutation product should be rendered substantially pure (i.e., as free of naturally occurring contaminants and LPS as is possible using available techniques known to and chosen by one of ordinary skill in the art).

The IMS of the invention may be used alone or may be incorporated in cis or in trans into a recombinant self-vector (plasmid, cosmid, virus or retrovirus) which may in turn code for any self- protein(s), -polypeptide(s), or -peptide(s) deliverable by a recombinant expression vector. For the sake of convenience, the IMSs are preferably administered without incorporation into an expression vector. However, if incorporation into an expression vector is desired, such incorporation may be accomplished using conventional techniques as known to one of ordinary skill in the art. For review those of ordinary skill would consult Ausubel, Current Protocols in Molecular Biology, supra. In some embodiments, an IMS can be co-administered with superphysiologic levels of one or divalent cations.

Briefly, construction of recombinant expression vectors employs standard ligation techniques. For analysis to confirm correct sequences in vectors constructed, the ligation mixtures may be used to transform a host T cell and successful transformants selected by antibiotic resistance where appropriate. Vectors from the transformants are prepared, analyzed by restriction and/or sequenced by, for example, the method of Messing et al., (Nucleic Acids Res., 9:309 (1981)), the method of Maxam et al. (Methods in Enzymology, 65:499 (1980)), or other suitable methods which will be known to those skilled in the art. Size separation of cleaved fragments is performed using conventional gel electrophoresis as described, for example, by Maniatis et al., (Molecular Cloning, pp. 133-134 (1982).

Host T cells may be transformed with the expression vectors of this invention and cultured in conventional nutrient media modified as is appropriate for inducing promoters, selecting transformants or amplifying genes. The culture conditions, such as temperature, pH and the like are those previously used with the host T cell selected for expression, and will be apparent to the ordinarily skilled artisan.

If a recombinant expression vector is utilized as a carrier for the IMS-ON of the invention, plasmids and cosmids are particularly preferred for their lack of pathogenicity. However, plasmids and cosmids are subject to degradation in vivo more quickly than viruses and therefore may not deliver an adequate dosage of IMS-ON to prevent or treat an inflammatory or autoimmune disease.

Most of the techniques used to construct vectors, and transfect and infect T cells, are widely practiced in the art, and most practitioners are familiar with the standard resource materials that describe specific conditions and procedures.

“Plasmids” and “vectors” are designated by a lower case p followed by letters and/or numbers. The starting plasmids are commercially available, publicly available on an unrestricted basis, or can be constructed from available plasmids in accord with published procedures. In addition, equivalent plasmids to those described are known in the art and will be apparent to the ordinarily skilled artisan. A “vector” or “plasmid” refers to a genetic element that is capable of replication by comprising proper control and regulatory elements when present in a host T cell. For purposes of this invention examples of vectors or plasmids include, but are not limited to, plasmids, phage, transposons, cosmids, virus, etc.

Construction of the vectors of the invention employs standard ligation and restriction techniques which are well understood in the art (see Ausubel et al., Current Protocols in Molecular Biology, (1987), Wiley-Interscience or Maniatis et al., Molecular Cloning: A laboratory Manual (Cold Spring Harbor Laboratory, N.Y.), (1992). Isolated plasmids, DNA sequences, or synthesized oligonucleotides are cleaved, tailored, and relegated in the form desired. The sequences of all DNA constructs incorporating synthetic DNA were confirmed by DNA sequence analysis (Sanger et al., Proc. Natl. Acad. Sci., 74:5463-5467 (1977)).

“Digestion” of DNA refers to catalytic cleavage of the DNA with a restriction enzyme that acts only at certain sequences, restriction sites, in the DNA. The various restriction enzymes used herein are commercially available and their reaction conditions, cofactors and other requirements are known to the ordinarily skilled artisan. For analytical purposes, typically 1 μg of plasmid or DNA fragment is used with about 2 units of enzyme in about 20 μl of buffer solution. Alternatively, an excess of restriction enzyme is used to insure complete digestion of the DNA substrate. Incubation times of about one hour to two hours at about 37° C. are workable, although variations can be tolerated. After each incubation, protein is removed by extraction with phenol/chloroform, and may be followed by ether extraction, and the nucleic acid recovered from aqueous fractions by precipitation with ethanol. If desired, size separation of the cleaved fragments may be performed by polyacrylamide gel or agarose gel electrophoresis using standard techniques. A general description of size separations is found in Methods of Enzymology, 65:499-560 (1980).

Restriction cleaved fragments may be blunt ended by treating with the large fragment of E. coli DNA polymerase I (Klenow) in the presence of the four deoxynucleotide triphosphates (dNTPs) using incubation times of about 15 to 25 minutes at 20 degree C. in 50 mM Tris (ph7.6) 50 mM NaCl, 6 mM mgCl2, 6 mM DTT and 5-10 mu.M dNTPs. The Klenow fragment fills in at 5′ sticky ends but chews back protruding 3′ single strands, even though the four dNTPs are present. If desired, selective repair can be performed by supplying only one of the dNTPs, or with selected dNTPs, within the limitations dictated by the nature of the sticky ends. After treatment with Klenow, the mixture is extracted with phenol/chloroform and ethanol precipitated. Treatment under appropriate conditions with S1 nuclease or Bal-31 results in hydrolysis of a single-stranded portion.

Ligations are performed in 15-50 μl volumes under the following standard conditions and temperatures: 20 mM Tris-Cl pH 7.5, 10 mM MgCl₂, 10 mM DTT, 33 mg/ml BSA, 10 mM-50 mM NaCl, and either 40 μm ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 33-100 μg/ml total DNA concentrations (5-100 mM total end concentration). Intermolecular blunt end ligations (usually employing a 10-30 fold molar excess of linkers) are performed at 1 μM total ends concentration.

The expression self-cassette will employ a promoter that is functional in host T cells. In general, vectors containing promoters and control sequences that are derived from species compatible with the host T cell are used with the particular host T cell. Promoters suitable for use with prokaryotic hosts illustratively include the beta-lactamase and lactose promoter systems, alkaline phosphatase, the tryptophan (trp) promoter system and hybrid promoters such as tac promoter. However, other functional bacterial promoters are suitable. In addition to prokaryotes, eukaryotic microbes such as yeast cultures may also be used. Saccharomyces cerevisiae, or common baker's yeast is the most commonly used eukaryotic microorganism, although a number of other strains are commonly available. Promoters controlling transcription from vectors in mammalian host T cells may be obtained from various sources, for example, the genomes of viruses such as: polyoma, simian virus 40 (SV40), adenovirus, retroviruses, hepatitis B virus and preferably cytomegalovirus, or from heterologous mammalian promoters, e.g., β-actin promoter. The early and late promoters of the SV40 virus are conveniently obtained as an SV40 restriction fragment which also contains the SV40 viral origin of replication. The immediate early promoter of the human cytomegalovirus is conveniently obtained as a HindIII restriction fragment. Of course, promoters from the host T cell or related species also are useful herein.

The vectors used herein may contain a selection gene, also termed a selectable marker. A selection gene encodes a protein, necessary for the survival or growth of a host T cell transformed with the vector. Examples of suitable selectable markers for mammalian cells include the dihydrofolate reductase gene (DHFR), the omithine decarboxylase gene, the multi-drug resistance gene (mdr), the adenosine deaminase gene, and the glutamine synthase gene. When such selectable markers are successfully transferred into a mammalian host T cell, the transformed mammalian host T cell can survive if placed under selective pressure. There are two widely used distinct categories of selective regimes. The first category is based on a cell's metabolism and the use of a mutant T cell line which lacks the ability to grow independent of a supplemented media. The second category is referred to as dominant selection which refers to a selection scheme used in any cell type and does not require the use of a mutant T cell line. These schemes typically use a drug to arrest growth of a host T cell. Those cells which have a novel gene would express a protein conveying drug resistance and would survive the selection. Examples of such dominant selection use the drugs neomycin (Southern and Berg, J. Molec. Appl. Genet., 1:327 (1982)), mycophenolic acid (Mulligan and Berg, Science, 209:1422 (1980)), or hygromycin (Sugden et al., Mol Cell. Bio., 5:410-413 (1985)). The three examples given above employ bacterial genes under eukaryotic control to convey resistance to the appropriate drug neomycin (G418 or genticin), xgpt (mycophenolic acid) or hygromycin, respectively.

Alternatively the vectors used herein are propagated in a host T cell using antibiotic-free selection based on repressor titration (Cranenburgh et al., 2001). The vectors are modified to contain the lac operon either as part of the lac promoter or with the lacO1 and lacO3 operators with the optimal spacing found in the pUC series of plasmid vectors. Alternatively the lacO1 operator or palindromic versions of the lacO can be used in isolation as single or multiple copies (Cranenburgh et al., 2004). The lac operon sequence may be incorporated at single or multiple sites anywhere within the vector so as not to interfere with other functional components of the vector. In preferred embodiments a synthetic Escherichia coli lac operon dimer operator (Genbank Acc. Num. K02913) is used. The lac operon may be added to a vector that lacks a suitable selective marker to provide selection, be added in addition to another selectable marker, or used to replace a selectable marker, especially an antibiotic resistance marker, to make the vector more suitable for therapeutic applications. Vectors containing the lac operon can be selected in genetically modified E. coli with an essential gene, including dapD, under the control of the lac promoter (lacOP) thus allowing the modified host T cell to survive by titrating the lac repression from the lacOP and allowing expression of dapD. Suitable E. coli stains include DH1lacdapD and DH1lacP2dapD (Cranenburgh et al., 2001)

One particularly suitable nucleic acid vector useful in accordance with the methods provided herein is a nucleic acid expression vector in which a non-CpG dinucleotide is substituted for one or more CpG dinucleotides of the formula 5′-purine-pyrimidine-C-G-pyrimidine-pyrimidine-3′ or 5′-purine-purine-C-G-pyrimidine-pyrimidine-3′, thereby producing a vector in which immunostimulatory activity is reduced. For example, the cytosine of the CpG dinucleotide can be substituted with guanine, thereby yielding an IMS region having a GpG motif of the formula 5′-purine-pyrimidine-G-G-pyrimidine-pyrimidine-3′ or 5′-purine-purine-G-G-pyrimidine-pyrimidine-3′. The cytosine can also be substituted with any other non-cytosine nucleotide. The substitution can be accomplished, for example, using site-directed mutagenesis. Typically, the substituted CpG motifs are those CpGs that are not located in important control regions of the vector (e.g., promoter regions). In addition, where the CpG is located within a coding region of an expression vector, the non-cytosine substitution is typically selected to yield a silent mutation or a codon corresponding to a conservative substitution of the encoded amino acid.

For example, in certain embodiments, the vector used for construction of the self-vector is a modified pVAX1 vector (SEQ ID NO: 1) in which one or more CpG dinucleotides of the formula 5′-purine-pyrimidine-C-G-pyrimidine-pyrimidine-3′ is mutated by substituting the cytosine of the CpG dinucleotide with a non-cytosine nucleotide. The pVAX1 vector is known in the art and is commercially available from Invitrogen (Carlsbad, Calif.). In one exemplary embodiment, the modified pVAX1 vector has the following cytosine to non-cytosine substitutions within a CpG motif: cytosine to guanine at nucleotides 784, 1161, 1218, and 1966; cytosine to adenine at nucleotides 1264, 1337, 1829, 1874, 1940, and 1997; and cytosine to thymine at nucleotides 1963 and 1987; with additional cytosine to guanine mutations at nucleotides 1831, 1876, 1942, and 1999. (The nucleotide number designations as set forth above are according to the numbering system for pVAX1 provided by Invitrogen.) The remaining four prototypical CpG elements in pVAX1 occur within important control regions of the vector, and were therefore left unmodified. The vector thus constructed was named BHT-1 (SEQ ID NO:2). Preparation and use of BHT-1 is described in WO 2004/047734.

In some embodiments, the present invention provides a self-vector comprising a BHT-1 expression vector backbone and a polynucleotide encoding a self-protein, -polynucleotide, or -peptide associated with multiple sclerosis. In certain embodiments the polynucleotide of the self-vector encodes human proteolipid protein (PLP). In other embodiments the polynucleotide of the self-vector encodes human myelin associated glycoprotein (MAG). In still other embodiments the polynucleotide of the self-vector encodes human myelin oligodendrocyte protein (MOG). In preferred embodiments the polynucleotide of the self-vector encodes human myelin basic protein (MBP). In a most preferred embodiment of the present invention, the self-vector is BHT-3009 (SEQ ID NO: 3), wherein BHT-3009 comprises a BHT-I expression vector backbone and a polynucleotide encoding human myelin basic protein.

“Transfection” means introducing DNA into a host T cell so that the DNA is expressed, whether functionally expressed or otherwise; the DNA may also replicate either as an extrachromosomal element or by chromosomal integration. Unless otherwise provided, the method used in examples herein for transformation of the host T cells is the calcium phosphate co-precipitation method of Graham and van der Eb, Virology, 52:456-457 (1973). Transfection may be accomplished by any method known in the art suitable for introducing an extracellular nucleic acid into a host T cell, including but not limited to, the use of transfection facilitating agents or processes such as calcium phosphate co-precipitation, zinc or other related metal cation-induced precipitates (metal cations generate sedimenting particles of phosphates or hydroxides for which DNA has a strong affinity, resulting in a DNA:metal phosphate co-sedimentation—requires submillimolar or millimolar concentrations of zinc or other metals (see Kejnovsky and Kypr, Nucleic Acids Research, 26:5295-99 (1998)), super-concentrated solutions to induce DNA precipitation, binding of DNA to gold or other particles, viral transduction, protoplast fusion, transfection mediated by DEAE-dextran or its analogs, polybrene-mediated transfection, liposome fusion, microinjection, microparticle bombardment (biolistics) or electroporation (Kriegler, Gene Transfer and Expression: A Laboratory Manual, Stockton Press (1990)).

In preferred embodiments the nucleic acid of interest is formulated with one or more divalent cations at a total concentration greater than physiological levels for injection into an animal for uptake by the host T cells of the animal. In some embodiments, one or more physiologically acceptable divalent cations can be used, e.g., Ca²⁺, Mg²⁺, Mn²⁺, Zn²⁺, Al²⁺, Cu²⁺, Ni²⁺, Ba²⁺, Sr²⁺, or others, and mixtures thereof. In some embodiments, the divalent cation is calcium alone. In some embodiments, magnesium, calcium or mixtures thereof, can be present extracellularly at approximately 1.5 mM and 1 mM, respectively. In preferred embodiments, the nucleic acid to be transfected is formulated with calcium at a concentration between about 0.9 mM (1×) to about 2 M; in more preferred embodiments the calcium concentration is between about 2 mM to about 8.1 mM (9×); in most preferred embodiments the calcium concentration is between about 2 mM to about 5.4 mM (6×). Mixtures of two or more divalent cations can be used in combinations amounting to total concentrations of about 0.9, 2, 4, 5, 6, 7, 8, 9, 10, 12, 15, 20, 45, 65, 90, 130, 170, 220, 280, 320, 350, 500, 750, 1000, 1500 mM, etc., and up to about 2 M.

In certain preferred embodiments, the counterion can include PO₄, Cl, OH, CO₂, or mixtures thereof. In other embodiments, the formulations may cause DNA to form particulate or precipitates with size distributions where the mean sizes, or the 80% particles, are in excess of about 0.1, 0.3, 0.5, 1, 3, 5, 8, 15, 20, 35, 50, 70 or 100 microns. Size of such particulates may be evaluated by centrifugation, flow cytometry analysis, propydium iodide or similar dye labeling, or dynamic light scattering.

Use of divalent cation(s) at a concentration greater than physiological levels is suitable for use with any DNA vaccination vector backbone. For the methods of the present invention, divalent cation(s) at a concentration greater than physiological levels also find use with any immunosuppressive vector backbone. Exemplified immunosuppressive vector backbones include those (i) with a reduced number of immunostimulatory sequences (ISS) in comparison to a parent vector backbone (e.g., a reduced number of “CpG” sequences), (ii) containing one or more immunoinhibitory sequences (IIS), and (iii) having a reduced number of ISS and one or more IIS. Exemplified immunosuppressive vector backbones include BHT-1 vector backbones.

Transformation methods are known in the art, and methods similar to that reported by Bishop (see Bio.com), Jordan et al. (1996) Nucleic Acids Research 15:24(4):596-601; U.S. Pat. No. 5,593,875; Chen and Okayama (1987) Mol. Cell Biol. 7(8):2745-2752; and Welzel, et al. (2004) “Transfection of cells with custom-made calcium phosphate nanoparticles coated with DNA” J. Mater. Chem. 14:2213-2217. Additional components may be used, e.g., histones, various salts, liposomes, charged entities such as polylysine, spermine, spermidine, and such. See, e.g., Simonson, et al. (2005) “Bioplex technology: novel synthetic gene delivery pharmaceutical based on peptides anchored to nucleic acids” Curr. Pharm. Des. 11(28):3671-680; Roche, et al. (2003) “Glycofection: facilitated gene transfer by cationic glycopolymers” Cell Mol. Life Sci. 60(2):288-297; Pichon, et al. (2001) “Histidine-rich peptides and polymers for nucleic acids delivery” Adv. Drug Deliv. Rev. 53(1):75-94; Mahat, et al. (1999) “.Peptide-based gene delivery” Curr. Opin. Mol. Ther. (2):226-243; and Lee and Kim (2005) “Polyethylene glycol-conjugated copolymers for plasmid DNA delivery” Pharm. Res. 22(1): 1 -10. See also, Pack, et al. (2005) “Design and Development of Polymers for Gene Delivery” Nature Drug Discovery 4:581-493.

The effectiveness of a particular divalent cation, a particular anion or counterion, combinations of mixtures of different divalent cations, and combinations of divalent cations and counterions can be measured on at least three different levels: (i) at the level of transfection, (ii) the level of expression (i.e., transcription or translation), and (iii) the level of immune response or immunosuppression. At the level of transfection, in vitro or in vivo transfection efficiency can be measured using any method known in the art (e.g., using quantitative PCR assays). At the level of expression, transcription or translation can be measured in vitro or in vivo using any method known in the art. For example, antibodies can be used to detect translation of a self-antigen or self-protein from cultured cells, or from target cells in vivo (e.g., muscle cells, dendritic cells, keratinocytes, fibroblasts, epithelial cells, and other target cell types or cells of target organs) in ELISA or Western Blot assays. At the level of the immune response, promotion, inhibition or prevention of an immune response resulting from such transfection or injection can be measured in vitro or in vivo using any method known in the art. For example, proliferation of activated lymphocytes, presence of autoreactive lymphocytes, production of autoantibodies, or cytokine production by lymphocytes or other immune cells (e.g. plasmacytoid dendritic cells) exposed to transfected target cells can be measured. Autoimmune disease symptoms (e.g., inflammation, tissue destruction, presence of autoantibodies or autoreactive lymphocytes), or amelioration thereof, in an animal model can also be measured after transfection or injection of a self-vector in superphysiological concentrations of one or more divalent cations. Animal models for numerous autoimmune diseases are described herein.

Self-vectors of this invention can be formulated as polynucleotide salts for use as pharmaceuticals. Polynucleotide salts can be prepared with non-toxic inorganic or organic bases. Inorganic base salts include sodium, potassium, zinc, calcium, aluminum, magnesium, etc. Organic non-toxic bases include salts of primary, secondary and tertiary amines, etc. Such self-DNA polynucleotide salts can be formulated in lyophilized form for reconstitution prior to delivery, such as sterile water or a salt solution. Alternatively, self-DNA polynucleotide salts can be formulated in solutions, suspensions, or emulsions involving water- or oil-based vehicles for delivery. In one preferred embodiment, the DNA is lyophilized in phosphate buffered saline with physiologic levels of calcium (0.9 mM) or another divalent cation, and then reconstituted with sterile water prior to administration. In some embodiments, the DNA is formulated in solutions containing higher than physiological quantities of one or more divalent cations, as described above, for example between 1 μM and 2 M total concentration of one or more divalent cations. In some embodiments, the DNA is formulated in solutions containing higher than physiological quantities of Ca++, for example, between 1 μM and 2 M. The DNA can also be formulated in the absence of specific ion species.

As known to those ordinarily skilled in the art, a wide variety of methods exist to deliver polynucleotide to subjects, as defined herein. “Subjects” shall mean any animal, such as, for example, a human, non-human primate, horse, cow, dog, cat, mouse, rat, guinea pig or rabbit. The polynucleotide encoding self-protein(s), -polypeptide(s), or -peptide(s) can be formulated with cationic polymers including cationic liposomes. Other liposomes also represent effective means to formulate and deliver self-polynucleotide. Alternatively, the self DNA can be incorporated into a viral vector, viral particle, or bacterium for pharmacologic delivery. Viral vectors can be infection competent, attenuated (with mutations that reduce capacity to induce disease), or replication-deficient. Particles also represent an effective method to deliver DNA, and DNA can be bound to gold or other particles follow by injection into the subject or delivered by a gene gun. Methods utilizing self-DNA to prevent the deposition, accumulation, or activity of pathogenic self proteins may be enhanced by use of viral vectors or other delivery systems that increase humoral responses against the encoded self-protein. In other embodiments, the DNA can be conjugated to solid supports including gold particles, polysaccharide-based supports, or other particles or beads that can be injected, inhaled, or delivered by particle bombardment (ballistic delivery).

Methods for delivering nucleic acid preparations are known in the art. See, e.g., U.S. Pat. Nos. 5,399,346, 5,580,859, 5,589,466. A number of viral based systems have been developed for transfer into mammalian cells. For example, retroviral systems have been described (U.S. Pat. No. 5,219,740; Miller et al., Biotechniques, 7:980-990 (1989); Miller, A. D., Human Gene Therapy, 1:5-14 (1990); Scarpa et al., Virology, 180:849-852 (1991); Bums et al., Proc. Natl. Acad. Sci. USA, 90:8033-8037 (1993); and Boris-Lawrie and Temin, Cur. Opin. Genet. Develop., 3:102-109 (1993)). A number of adenovirus vectors have also been described (see, e.g., Haj-Ahmad et al., J. Virol., 57:267-274 (1986); Bett et al., J. Virol., 67:5911-5921 (1993); Mittereder et al., Human Gene Therapy, 5:717-729 (1994); Seth et al., J. Virol., 68:933-940 (1994); Barr et al., Gene Therapy, 1:51-58 (1994); Berkner, K. L., BioTechniques, 6:616-629 (1988); and Rich et al., Human Gene Therapy, 4:461-476 (1993)). Adeno-associated virus (AAV) vector systems have also been developed for nucleic acid delivery. AAV vectors can be readily constructed using techniques well known in the art (see, e.g., U.S. Pat. Nos. 5,173,414 and 5,139,941; International Publication Nos. WO 92/01070 and WO 93/03769; Lebkowski et al., Molec. Cell. Biol., 8:3988-3996 (1988); Vincent et al., Vaccines, 90 (Cold Spring Harbor Laboratory Press) (1990); Carter, B. J., Current Opinion in Biotechnology, 3:533-539 (1992); Muzyczka, N., Current Topics in Microbiol. And Immunol., 158:97-129 (1992); Kotin, R. M., Human Gene Therapy, 5:793-801 (1994); Shelling et al., Gene Therapy, 1:165-169 (1994); and Zhou et al., J. Exp. Med., 179:1867-1875 (1994)).

The polynucleotide of this invention can also be delivered without a viral vector. For example, the molecule can be packaged in liposomes prior to delivery to the subject. Lipid encapsulation is generally accomplished using liposomes which are able to stably bind or entrap and retain nucleic acid. For a review of the use of liposomes as carriers for delivery of nucleic acids (see Hug et al., Biochim. Biophys. Acta., 1097:1-17 (1991); Straubinger et al., Methods of Enzymology, 101:512-527 (1983)). See also, Pack, et al. (2005) “Design and Development of Polymers for Gene Delivery” Nature Drug Discovery 4:581-493.

“Treating,” “treatment,” or “therapy” of a disease or disorder shall mean slowing, stopping or reversing the disease's progression, as evidenced by cessation or elimination of either clinical or diagnostic symptoms, by administration of a polynucleotide encoding a self-protein(s), -polypeptide(s) or -peptide(s) either alone or in combination with another compound as described herein. In the preferred embodiment, treating a disease means reversing or stopping the disease's progression, ideally to the point of eliminating the disease itself. As used herein, ameliorating a disease and treating a disease are equivalent.

“Preventing,” “prophylaxis” or “prevention” of a disease or disorder as used in the context of this invention refers to the administration of a polynucleotide encoding a self-protein(s), -polypeptide(s), or -peptide(s) either alone or in combination with another compound as described herein, to prevent the occurrence or onset of a disease or disorder or some or all of the symptoms of a disease or disorder or to lessen the likelihood of the onset of a disease or disorder.

“Therapeutically effective amounts” of the self-vector comprising polynucleotide encoding one or more self-protein(s), -polypeptide(s) or -peptide(s) is administered in accord with the teaching of this invention and will be sufficient to treat or prevent the disease as for example by ameliorating or eliminating symptoms and/or the cause of the disease. For example, therapeutically effective amounts fall within broad range(s) and are determined through clinical trials and for a particular patient is determined based upon factors known to the ordinarily skilled clinician including the severity of the disease, weight of the patient, age and other factors. Therapeutically effective amounts of self-vector are in the range of about 0.001 micrograms to about 1 gram. A preferred therapeutic amount of self-vector is in the range of about 10 micrograms to about 5 milligrams. A most preferred therapeutic amount of self-vector is in the range of about 0.025 mg to 5 mg. Polynucleotide therapy is delivered monthly for 6-12 months, and then every 3-12 months as a maintenance dose. Alternative treatment regimens may be developed and may range from daily, to weekly, to every other month, to yearly, to a one-time administration depending upon the severity of the disease, the age of the patient, the self-protein(s), -polypeptide(s) or -peptide(s) being administered and such other factors as would be considered by the ordinary treating physician.

In one embodiment the polynucleotide is delivered by intramuscular injection. In another embodiment the polynucleotide is delivered intranasally, orally, subcutaneously, intradermally, intravenously, mucosally, impressed through the skin, or attached to gold particles delivered to or through the dermis (see, e.g., WO 97/46253). Alternatively, nucleic acid can be delivered into skin cells by topical application with or without liposomes or charged lipids (see, e.g., U.S. Pat. No. 6,087,341). Yet another alternative is to deliver the nucleic acid as an inhaled agent.

The polynucleotide can be formulated in phosphate buffered saline with physiologic levels of calcium (0.9 mM) and is endotoxin-free. Alternatively, the polynucleotide can be formulated or co-administered in solutions containing one or more divalent cations, for example, Ca²⁺, Mn²⁺, Mn²⁺, Zn²⁺, Al²⁺, Cu²⁺, Ni²⁺, Ba²⁺, Sr²⁺, and mixtures thereof, at higher than physiologic concentrations, for example, between 2 mM and 2 M, as discussed herein. Improved efficiency of one or more of transfection, autoantigen expression and improved therapeutic efficacy can be achieved when the self-vector and the one or more cations are co-administered at the same time or are administered sequentially. When administered sequentially, either the self-vector or the one or more divalent cations can be administered first.

Alternatively, or in addition, the polynucleotide may be formulated either with a cationic polymer, cationic liposome-forming compounds, or in non-cationic liposomes. Examples of cationic liposomes for DNA delivery include liposomes generated using 1,2-bis(oleoyloxy)-3-(trimethylammionio) propane (DOTAP) and other such molecules.

Prior to delivery of the polynucleotide, the delivery site can be preconditioned by treatment with bupivicane, cardiotoxin or another agent that may enhance the delivery of subsequent polynucleotide therapy. Such preconditioning regimens are generally delivered 12 to 96 hours prior to delivery of therapeutic polynucleotide, more frequently 24 to 48 hours prior to delivery of the therapeutic DNA. Alternatively, no preconditioning treatment is given prior to DNA therapy. In some embodiments, the delivery site is preconditioned with the administration of one or more divalent cations at greater than physiologic concentrations.

The self-vector can be administered in combination with other substances, such as, for example, pharmacological agents, adjuvants, cytokines, or vectors encoding cytokines. Furthermore, to avoid the possibility of eliciting unwanted anti-self cytokine responses when using cytokine codelivery, chemical immunomodulatory agents such as the active form of vitamin D3 can also be used. In this regard, 1,25-dihydroxy vitamin D3 has been shown to exert an adjuvant effect via intramuscular DNA immunization.

A polynucleotide coding for a protein known to modulate a host's immune response (e.g., an cytokine) can be coadministered with the self vector. Accordingly, a gene encoding an immunomodulatory cytokine (e.g., an interleukin, interferon, or colony stimulating factor), or a functional fragment thereof, may be used in accordance with the instant invention. Gene sequences for a number of these cytokines are known. Thus, in one embodiment of the present invention, delivery of a self-vector is coupled with coadministration of at least one of the following immunomodulatory proteins, or a polynucleotide encoding the protein(s): IL-4; IL-10; IL-13; TGF-beta; or IFN-gamma.

Nucleotide sequences selected for use in the present invention can be derived from known sources, for example, by isolating the nucleic acid from cells containing a desired gene or nucleotide sequence using standard techniques. Similarly, the nucleotide sequences can be generated synthetically using standard modes of polynucleotide synthesis that are well known in the art (see, e.g., Edge et al., Nature, 292:756 (1981); Nambair et al., Science, 223:1299 (1984); (Jay et al., J. Biol. Chem., 259:6311 (1984)). Generally, synthetic oligonucleotides can be prepared by either the phosphotriester method as described by (Edge et al., supra) and (Duckworth et al., Nucleic Acids Res., 9:1691 (1981)), or the phosphoramidite method as described by (Beaucage et al., Tet. Letts., 22:1859 1981), and (Matteucci et al., J. Am. Chem. Soc., 103:3185 (1981)). Synthetic oligonucleotides can also be prepared using commercially available automated oligonucleotide synthesizers. The nucleotide sequences can thus be designed with appropriate codons for a particular amino acid sequence. In general, one will select preferred codons for expression in the intended host. The complete sequence is assembled from overlapping oligonucleotides prepared by standard methods and assembled into a complete coding sequence. See, e.g., Edge et al. (supra); Nambair et al. (supra) and Jay et al. (supra).

Another method for obtaining nucleic acid sequences for use herein is by recombinant means. Thus, a desired nucleotide sequence can be excised from a plasmid carrying the nucleic acid using standard restriction enzymes and procedures. Site specific DNA cleavage is performed by treating with the suitable restriction enzymes and procedures. Site specific DNA cleavage is performed by treating with the suitable restriction enzyme (or enzymes) under conditions which are generally understood in the art, and the particulars of which are specified by manufacturers of commercially available restriction enzymes. If desired, size separation of the cleaved fragments may be performed by polyacrylamide gel or agarose gel electrophoreses using standard techniques.

Yet another convenient method for isolating specific nucleic acid molecules is by the polymerase chain reaction (PCR). (Mullis et al., Methods Enzymol., 155:335-350 (1987) or reverse transcription PCR (RT-PCR)). Specific nucleic acid sequences can be isolated from RNA by RT-PCR. RNA is isolated from, for example, cells, tissues, or whole organisms by techniques known to one skilled in the art. Complementary DNA (cDNA) is then generated using poly-dT or random hexamer primers, deoxynucleotides, and a suitable reverse transcriptase enzyme. The desired polynucleotide can then be amplified from the generated cDNA by PCR. Alternatively, the polynucleotide of interest can be directly amplified from an appropriate cDNA library. Primers that hybridize with both the 5′ and 3′ ends of the polynucleotide sequence of interest are synthesized and used for the PCR. The primers may also contain specific restriction enzyme sites at the 5′ end for easy digestion and ligation of amplified sequence into a similarly restriction digested plasmid vector.

The following examples are specific embodiments for carrying out the present invention. The examples are offered for illustrative purposes only, and are not intended to limit the scope of the present invention in any way.

### EXAMPLES

### Example 1

### DNA Particle Sizing

DNA samples (BHT-3021) were obtained on dry ice from Bayhill Therapeutics and were stored at −80° C. until further use. The DNA sample concentration was 2 mg/ml. The dynamic light scattering analysis was performed at two different DNA concentrations in the presence and absence of calcium chloride. Four different concentrations (0.9, 3, 5.4 and 8 mM) of calcium chloride were used for the analyses. The stock solution of DNA was diluted in phosphate buffered saline to obtain two different concentrations of DNA (0.25 and 1.5 mg/ml). The hydrodynamic diameter of the DNA samples was measured at 20° C. using a light scattering instrument (Brookhaven Instruments Corp, Holtszille, N.Y.) equipped with a 50 mW diode-pumped laser (λ=532 nm) incident upon a sample cell immersed in a bath of decalin. The scattered light was monitored by a PMT (EMI 9863) at 90° to the incident beam and the autocorrelation function was generated by a digital correlator (BI-9000AT). Data were collected continuously for five 30-seconds intervals for each sample and averaged. Data was analyzed by a variety of methods to yield information about the polydispersity of the preparation and the relative sizes of the various components present. The autocorrelation function was fit by the method of cumulants to yield the average diffusion coefficient of the DNA and/or complexes. The effective hydrodynamic diameter was obtained from the diffusion coefficient by the Stokes-Einstein equation. In addition, the data was fit to a non-negatively constrained least squares algorithm to yield multi-modal distributions. Also, for a more complete analysis, these methods were employed using a number average and an intensity average of the population.

### Particle Size Analysis by Particle Counting Machines

Experimental: A Coulter Multisizer 3 (Beckman Coulter Inc.) with an overall sizing range of 0.4-1200 μm was employed to perform an analysis of the aggregation state of DNA/Ca-phosphate complexes. A 560 μm aperture tube was used for all the DNA samples.

### Example 2

### Treatment of Multiple Sclerosis with BHT-3009 to Establish Safety and Preliminary Evaluation of Immune Response to hMBP

Currently approved agents for treating MS are non-specific immunomodulators. Acute relapses are typically managed with short-term courses of high dose corticosteroid therapy, which accelerates the rate of improvement after acute relapse but does not clearly improve overall recovery compared to placebo (Brusaferri et al., J. Neurol., 247:435-42 (2000)). Immunomodulating agents used to reduce the frequency and severity of attacks include interferon Beta 1B (Betaseron, Berlex), interferon Beta 1A (Avonex, Biogen; Rebif, Serono), glatiramer acetate (Copaxone, Teva Neuroscience), natalizumab (Tysabri, Biogen-Idec) and mitoxantrone (Novantrone, Amgen). None of these agents, however, address the underlying autoimmune response directly. Rather, they modulate one or more effector pathways shared by normal immunological processes that lead to disease related tissue damage. Furthermore, the effects of these products on disease progression are modest at best (Goodin et al., Neurol., 58:169-78 (2002); Filippini et al., Lancet, 361:545-52 (2003); Scott & Friggitt, CNS Drugs, 18:379-96 (2004); Simpson et al., CNS Drugs, 16:825-50 (2002); Miller et al., N. Engl. J. Med., 348:15-23 (2003)), and all have significant side effects. Specifically the interferons frequently cause flu-like symptoms in patients (Goodin et al., Neurol., 58:169-78 (2002); Filippini et al., Lancet, 361:545-52 (2003)); mitoxantrone causes myelosuppression with increased risk for infections (Scott & Friggitt, CNS Drugs, 18:379-96 (2004)); glatiramer acetate causes allergic reactions (Simpson et al., CNS Drugs, 16:825-50 (2002)), and Tysabri decreases lymphocyte trafficking (Miller et al., N. Engl. J. Med., 348:15-23 (2003)) and may increase the risk for infections including progressive multifocal leukoencephalopathy. In contrast to these non-specific immune inhibitors, BHT-3009 is designed to decrease selectively the immune response to myelin basic protein. It is hoped that antigen-specific immunosuppression will be more effective and safer than current therapies.

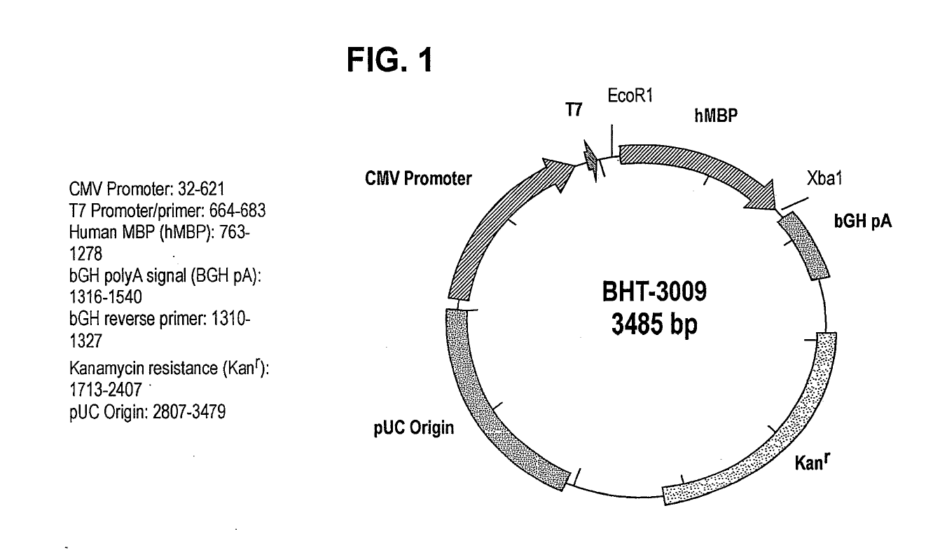

MS patients were enrolled in a multi-center, randomized, double-blind, three-arm, placebo-controlled phase I clinical trial to evaluate the safety of immunotherapy with BHT-3009 (SEQ ID NO:3) alone or in combination with atorvastatin. BHT-3009 is a plasmid vector comprising a BHT-1 expression vector backbone and a polynucleotide encoding full-length human myelin basic protein (hMBP) inserted into the EcoRI and Xba I sites within the multiple cloning sequence of BHT-1. Important functional and control features of BHT-3009 include a human cytomegalovirus (CMV) immediate-early gene promoter/enhancer; a bovine growth hormone gene polyadenylation signal; a kanamycin resistance gene; and a pUC origin of replication for propagation of the vector in E. coli. A diagram showing the main structural features of BHT-3009 is shown in FIG. 1. Intramuscular administration of BHT-3009 results in transient, low-level expression of hMBP protein at the injection site and also within cells that traffic to draining lymph nodes. This limited expression of a self-antigen in a novel immunological context has been demonstrated to attenuate ongoing autoimmune responses in mouse and rat models of experimental autoimmune encephalomyelitis, preclinical models for MS. The target population for this study was patients with relapsing disease including patients with relapsing remitting MS (RRMS) and a relatively stable course and patients with secondary progressive MS (SPMS) with relapses and a relatively stable course. Specific inclusion and exclusion criteria were as follows:

### Inclusion Criteria:

Definitive diagnosis of multiple sclerosis according to the McDonald criteria
Relapsing disease as shown by one or more of the following: acute relapse within previous two years; clinical deterioration over previous two years; gadolinium enhancing lesions on MRI
Clinically stable for >3 months.
At least one gadolinium enhancing lesion on brain MRI
Off interferon for >3 months before baseline evaluation.
Off immunosuppressive and cytotoxic therapy (e.g. mitoxantrone, cladrabine) >12 months or >6 months with CD4 count >400.
EDSS<7
Age>18 years.
Able to give informed consent.
WBC and platelets in normal range, hemoglobin >10.0 g/dl.
AST, ALT, bili<upper limit of normal.
Creatinine<upper limit of normal.

### Exclusion Criteria:

High-dose corticosteroids (e.g. >500 mg methylprednisolone or equivalent) within previous three months.
Previous therapy with vaccine therapy, stem cell transplantation or total lymphoid radiation at any time or glatiramer therapy within the previous 12 months.
Pregnant or lactating women
Unwilling to use a medically acceptable form of birth control
Known or suspected infection with HIV, hepatitis B or hepatitis C
Clinically significant ECG abnormalities
Medical condition or social circumstances that would in the opinion of the investigator prevent full participation in the trial or evaluation of study endpoints.
Implanted pace makers, defibrillators or other metallic objects on or inside the body that limit performing MRI scans.

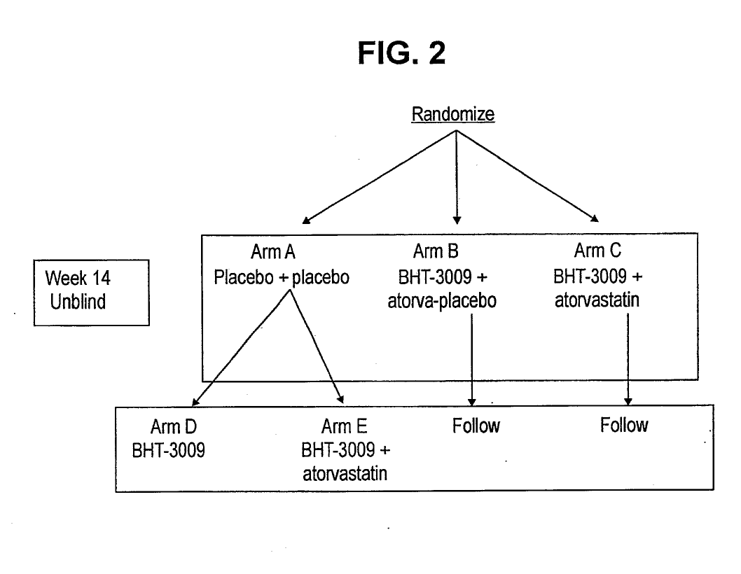

Thirty MS patients were assigned to one of three BHT-3009 dose cohorts. For each dose cohort, 10 patients were randomized into one of the following treatment arms: Arm A: BHT-placebo+atorvastatin-placebo (4 patients); Arm B: BHT-3009+atorvastatin-placebo (3 patients); and Arm C: BHT-3009+atorvastatin (3 patients). Patients randomized to Arm A were re-randomized to open-label treatment with one of the following: Arm D: BHT-3009 alone (2 patients) or Arm E: BHT-3009+atorvastatin (2 patients) and were treated and evaluated as patients originally randomized to Arms B or C, respectively, as described below (FIG. 2). All patients were evaluated in weeks -2 to 0 for baseline observations including MRI with gadolinium. At week 0 patients were randomized with treatment began in week 1. BHT-3009 and BHT-placebo were administered intramuscularly (IM) in weeks 1, 3, 5 and 9 at 0.5 mg, 1.5 mg and 3.0 mg doses. The BHT-3009 active biologic was produced in compliance with GMP standards. The final formulation of BHT-3009 was a sterile endotoxin-free, isotonic solution at 1.5 mg/mL in PBS containing 0.9 mM calcium (1×). In other embodiments of the present invention, BHT-3009 is formulated with a divalent cation such as calcium at a concentration between about 2 mM to about 2 M; in more preferred embodiments the calcium concentration is between about 2 mM to about 8.1 mM (9×); in most preferred embodiments the calcium concentration is between about 2 mM to about 5.4 mM (6×). BHT-placebo is a sterile, endotoxin-free, isotonic solution in PBS with calcium at 0.9 mM. Atorvastatin (Lipitor®) and atorvastatin-placebo were taken daily orally as 80 mg tablets beginning 2 days before the first BHT-3009/BHT-placebo injection and continued until the treatment was unblinded. MRI and other safety evaluations were performed at baseline and in weeks 5 and 9. In week 13, each patient underwent complete evaluation after which the treatment blind was broken. Patients randomized to Arms B and C stopped all protocol-specific therapy at week 14 and were followed for safety in weeks 26, 38 and 50.

TABLE 3
BHT-3009 and Atorvastatin Doses

| | | | |
|---|---|---|---|
| Dose Level | No. Patients | BHT-3009 Dose | Atorvastatin dose |

| | | | |
|---|---|---|---|
| 1 | 10 | 500 ug | 80 mg |
| 2 | 10 | 1500 ug | 80 mg |
| 3 | 10 | 3000 ug | 80 mg |

TABLE 4
Summary of the Schedule of Treatments and Evaluation

All Patients
Weeks −2 to 0: Baseline observations including MRI with gadolinium
Week 0: Randomization
Arms A, B or C
Weeks 1, 3, 5, 9: BHT-3009/BHT-placebo injections
Weeks 1-14 (unblinding): Daily atorvastatin/atorva-placebo tablets
Weeks 5 & 9: MRI with gadolinium, interim safety evaluation
Week 13: Full safety evaluation
Week 14: Unblind, re-randomize Arm A patients
Arm A Patients Re-Randomized to Arms D or E
Week 14, 16, 18, 22: BHT-3009 injections - open label
Weeks 14-26: Daily atorvastatin (Arm E patients only)
Weeks 18 & 22: MRI with gadolinium, interim safety evaluation
Week 26: Full safety evaluation
Weeks 38, 50 & 62: Full safety evaluation
Arm B & C Patients
Weeks 26, 38 & 50: Full safety evaluation

The following safety variables were evaluated:

### Clinical

History and physical including complete neurological exam
Problem-oriented history and physical exam
Vital signs
Concomitant medications
Injection site(s) evaluation
Kurtzke Expanded Disability Status Scale (EDSS)

### Laboratory

Chemistries (expanded): Glucose, BUN, creatinine, AST, ALT, alkaline phosphatase, total bilirubin, electrolytes (sodium, potassium, chloride, bicarbonate, calcium and magnesium), LDH, amylase, albumin, total protein.
Chemistries: Glucose, BUN, creatinine, AST, ALT, alkaline phosphatase, total bilirubin.
ANA, anti-DNA antibodies
Serum creatine kinase
Cholesterol.
CBC: Hematocrit, hemoglobin, WBC with differential (automated), platelets
Urinalysis: Dip stick plus microscopic examination if clinically significant abnormalities on dip stick
Urine pregnancy test for women of child-bearing potential only
Optional lumbar puncture for oligoclonal bands and IgG index, cell count and protein level
SPEP (serum protein electrophoresis)—only if LP performed
EKG—12 lead with rhythm strip

### Radiographic

Chest PA and Lateral
Magnetic resonance imaging (MRI) of the brain with gadolinium enhancement

### Special Tests

Vector expression in blood
MBP protein in blood

Preliminary safety data for the first ten subjects revealed two serious adverse events.

While one event was not study drug related, the other event, worsening depression in a subject with pre-existing depression, was considered to be possibly treatment-related. All other study drug-related adverse events were mild/moderate in severity with similar incidences in the placebo and study drug arms. Specifically, mild immediate injection site reactions were observed with similar frequency after injection of placebo (n=2) and BHT-3009 (erythema, n=1). No delayed injection site reactions suggestive of delayed hypersensitivity reactions were observed. Furthermore, there were no immediate systemic reactions suggestive of allergic reactions and no notable delayed systemic reactions after the study. There were three BHT-3009 related adverse events: diarrhea, dyspepsia and night sweats all of which were transient grade 1 events. There were no clinically-significant laboratory abnormalities related to BHT-3009.

In addition to safety the following immune response variables were evaluated: 1) T cell proliferation and intracellular cytokine production to specific antigens including MBP, PLP, MOG, tetanus and glatiramer acetate; 2) B cell antibody responses to specific antigens including MBP, PLP and MOG; 3) peripheral blood mononuclear cell (PBMC) phenotype assessed by flow cytometry; and 4) whole blood markers of inflammation assessed by quantitative PCR. For most assays, cell and serum samples were collected and stored until subjects had completed the treatment. Preliminary results indicate that the subjects treated with BHT-3009 showed a Th1 response to MBP as indicated by cell proliferation to MBP by CSFE dye dilution assay and production of IFNgamma by intracellular cytokine staining.

BHT-3009 was safe, well-tolerated, provided favorable trends on brain MRI, and produced beneficial antigen-specific immune changes. These immune changes consisted of a marked decrease in proliferation of interferon-gamma producing myelin-reactive CD4+T cells from peripheral blood, and a reduction in titers of myelin specific autoantibodies from cerebral spinal fluid as assessed by protein microarrays. We did not observe a substantial benefit of the atorvastatin combination compared to BHT-3009 alone.

In MS patients, BHT-3009 is safe and induces antigen-specific immune tolerance with concordant reduction of inflammatory lesions on brain MRI.

### Example 3

### Treatment of Multiple Sclerosis with BHT-3009 to Evaluate Reduction in CNS Inflammation

MS patients will be enrolled in a multi-center, randomized, double-blind, placebo-controlled phase 2b clinical trial to evaluate the safety, tolerability and efficacy of BHT-3009. Efficacy will be evaluated by reductions in CNS inflammation as assessed by gadolinium-enhanced lesions and other MRI measures that are indicators of possible clinical benefit. A positive outcome will support performing additional trials that test BHT-3009's clinical efficacy directly. This trial will also seek preliminary evidence for clinical efficacy (i.e. reduction in relapses and improved functional scores) although the trial is not adequately powered for this secondary purpose.

The target population for this trial is subjects with relapsing remitting MS who have EDSS<3.5 and have received less than six months of treatment with disease modifying agents who are most likely to benefit from antigen-specific immunotherapy. Specific inclusion and exclusion criteria are as follows: xxx

### Inclusion Criteria:

Definite diagnosis of MS by the McDonald criteria (34).
Screening cranial MRI demonstrating lesions consistent with MS.
One or more relapses within the previous year.
Clinically stable (no relapses) for >50 days before beginning screening procedures and during the screening period.
EDSS 0 to 3.5 inclusive.
Age >18 years and <55 years.
Willing and able to give informed consent.
WBC >3,000; platelets >100,000; hemoglobin >10.0 g/dl
AST, ALT, bilirubin <2.0×upper limit of normal
Creatinine <2.0×upper limit of normal.
Negative test for HIV.

### Exclusion Criteria:

Primary progressive, secondary progressive or progressive relapsing MS.
More than fifteen gadolinium-enhancing on the first screening MRI.
High-dose corticosteroids (e.g. >500 mg methylprednisolone or equivalent per day for 3 or more days) within 50 days prior to beginning screening procedures.
Previous stem cell transplantation, total lymphoid radiation, or cytotoxic therapy.
Treatment with interferon, glatiramer acetate or other approved disease-modifying agents for >180 days (lifetime total of all agents).
Treatment with an approved disease modifying agent within 180 days of beginning screening procedures.
Previous treatment of MS with an experimental agent including off-label use of approved drugs. (Allowed with approval of the Medical Monitor.)
Prior therapy with natalizumab (Tysabri)
Pregnant or lactating women.
Unwilling to use a medically acceptable form of birth control (e.g. hormonal contraception, intrauterine device, double barriers, sterilization of self or partner).
Clinically significant ECG abnormalities (e.g. acute ischemia or life-threatening arrhythmia).
Medical condition or social circumstances that would in the opinion of the investigator prevent full participation in the trial or evaluation of study endpoints.
Implanted pace makers, defibrillators or other metallic objects on or inside the body that limit performing MRI scans.
Known hypersensitivity or allergy to gadolinium.

Eligible patients (n=252) will be randomized in equal numbers to three arms: Arm A: 0.5 mg BHT-3009; Arm B: 1.5 mg BHT-3009; and Arm C: BHT-placebo. The BHT-3009 active biologic is produced in compliance with GMP standards. The final formulation of BHT-3009 is a sterile endotoxin-free, isotonic solution at 1.5 mg/mL in PBS containing 0.9 mM calcium (1×). In other embodiments of the present invention, BHT-3009 is formulated with a divalent cation such as calcium at a concentration between about 0.05 mM to about 2 M; in more preferred embodiments the calcium concentration is between about 2 mM to about 8.1 mM (9×); in most preferred embodiments the calcium concentration is between about 2 mM to about 5.4 mM (6×). Study drug will be administered intramuscularly at weeks 0, 2, 4, and then every 4 weeks through week 44 inclusive for a total of 13 doses. Study drug will be administered via two syringes at two separate injection sites with 0.33 mL in syringe #1 and 0.67 mL in syringe #2. The arms are the preferred injection site because of the extensive lymph node drainage from the arms. If injection into the deltoids is not possible, then injection into the second or third choice sites is acceptable. Second choice injections sites are the anterior thighs in the middle of the quadriceps muscle, and third choice sites are the buttocks.

TABLE 5
BHT-3009 Doses

| | | Study Vial #1 | | Study Vial #2 | |
|---|---|---|---|---|---|
| | | Contents | Volume | Contents | Volume |
| Study Arm | Dose | (Blinded) | injected | (Blinded) | injected |
| Arm A | 0.5 mg | BHT-3009 | 0.33 mL | Placebo | 0.67 mL |
| Arm B | 1.5 mg | BHT-3009 | 0.33 mL | BHT-3009 | 0.67 mL |
| Arm C | Placebo | Placebo | 0.33 mL | Placebo | 0.67 mL |

The primary endpoint is the mean four-week rate of occurrence of new Gd-enhancing lesions on cranial MRIs performed every 4 weeks from week 28 through week 48 (6 MRIs total). Secondary endpoints include the following:

MRI
T2 lesion volume change from baseline to Week 48.
Mean 4 week rate of new T2 lesions on the cranial MRIs performed every 4 weeks from Week 28 through Week 48.
T1 hypointense lesion volume change and chronic T1 hypointense lesion volume change from baseline to Week 48.
Mean Gd-enhancing lesion volume on cranial MRIs performed from Week 28 through Week 48.

Relapses
Annualized rate of relapses.
Time to first relapse, censoring subjects who withdraw.

Functional Scores (EDSS & MSFC)
The proportion of subjects with worsening EDSS on Week 48 evaluation compared to baseline.
The proportion of subjects with confirmed worsening MSFC on Week 48 evaluation compared to baseline.

MRI will be performed twice during screening and at weeks 8, 16, 28, 32, 36, 40, 44 and 48. All images for this trial will be acquired on a 1.5 Tesla or greater magnet unless approved by the Sponsor with a customized set of sequence parameters worked out for each site during a dummy run. Subjects will have their MRI scans performed on the same scanner using the same sequences to include complete brain coverage, minimal subject motion and consistency over time. Contrast will be given at a dose standard for the study. One to three dummy MRIs will be performed on volunteers to demonstrate adequate image quality and to establish procedures for transmission and data management.

Relapses will be assessed as soon as possible after they occur and must be confirmed by the examining physician. A relapse is defined as the appearance or reappearance of one or more significant neurological abnormalities persisting for at least 48 hours and immediately preceded by a period of relatively stable or improving disease for at least 30 days. Normal fluctuations in a subject's MS symptoms do not themselves constitute a relapse, and appearance or reappearance of neurological abnormalities with an apparent precipitating event such as an infection or fever will not be considered a relapse. A relapse will be considered confirmed when the subject's symptoms are accompanied by objective changes on the neurological examination and an increase in Kurtzke's Expanded Disability Status Score (EDSS) of at least 1.0 point. A change in bowel/bladder function, change in severity of a pre-existing somatosensory defect or change in cognitive function will not be solely responsible for a confirmed relapse.

Disability status will be assessed using two different routine research assessment criteria: Kurtzke's Expanded Disability Status Score (EDSS; Kurtzke, Neurol., 33:1444-52 (1983)) and Multiple Sclerosis Functional Composite score (MSFC; Cutter et al., Brain, 122:871-82 (1999)) assessments. EDSS and MSFC will be performed during screening and at weeks 40 and 48. EDSS will be performed by an “Examining Physician” who is not the “Treating Physician” and is blinded to the subject's clinical status. MSFC may be performed by qualified trained clinic staff, the Treating Physician or the Examining Physician. Worsening EDSS at week 48 is defined as an initial increase in EDSS consistent with worsening at week 40 that is confirmed 8 weeks later at week 48. Subjects who are experiencing a relapse are not considered to have worsening EDSS until their condition has stabilized. Worsening MSFC is defined as a one unit or greater decrease in MSFC z-score confirmed at least 8 weeks later. Worsening MSFC in week 48 is defined as a one unit or greater decrease in z-score in week 40 compared to screening MSFC z-score that is confirmed in week 48. Subjects who are experiencing a relapse are not considered to have worsening MSFC until their condition has stabilized.

The primary test of the superiority of either of two the doses of BHT-3009 to placebo will be performed by examining differences between treatment groups in the primary variable using a generalized linear model assuming the Poisson distribution and using the log link function on the ITT population, with treatment group and pooled center as factors and the log of the number of gadolinium (Gd) enhancing lesions on the baseline MRI scan as covariate. Where the number of lesions at baseline is zero, this will be approximated by log(0.1). Overdispersion will be taken account of and will be estimated via the deviance. The superiority of BHT-3009 to placebo will be examined via null hypotheses of the form: H0: BHT-3009 does not differ from placebo versus Hi: BHT-3009 differs from placebo. The two null hypotheses with their corresponding alternatives will each specify a different dose of BHT-3009: 0.5 mg and 1.5 mg. The null hypotheses will be examined via Wald chi-square tests of the estimates of differences in least-squares means of the treatment groups. These estimates will be presented, together with their 95% confidence intervals (CIs). Hochberg's multiple test procedure will be employed to account for multiplicity in the calculation of CIs. The primary variable is assumed to follow the Poisson distribution with overdispersion estimated by the deviance. Goodness of fit of the model will be assessed using the Hosmer-Lemeshow statistic for goodness of fit. Validity of the assumptions may also be assessed visually, using Q-Q plots. If the Poisson distribution is clearly not applicable, a 2-sided Wilcoxon test will be performed, stratified by pooled center and number of Gd+lesions on baseline MRI scan (0, 1-5, >5 lesions); and unstratified Hodges-Lehmann estimates of treatment difference and their CIs will be presented.

289 patients were randomized. 272 patients completed the planned 44 weeks of treatment. Treatment has been well tolerated. 199 patients (68.9%) reported one or more treatment-emergent adverse events (AEs) so far. In only 44 patients (15.2%) are these AEs felt to be possibly related and in 39 patients (13.5%) probably related to study drug. Most AEs were mild/moderate in severity. There have been no significant clinical laboratory abnormalities to date. There were no imbalances in AEs across the three treatment arms. Baseline ELISPOT assays on 77 patients demonstrated that 63 patients (81.8%) were positive for interferon-gamma production to one or more MBP peptides, 58 (75.3%) were positive for PLP peptides, and 53 (68.8%) were positive for MOG peptides. Follow up ELISPOT and CSF assays are being performed at week 44.

The data from the phase I/II trial suggest that BHT-3009 is safe and may suppress immune responses in an antigen-specific manner.

### Example 4

### Characterization of the Activity of BHT-3021 High Calcium Formulations

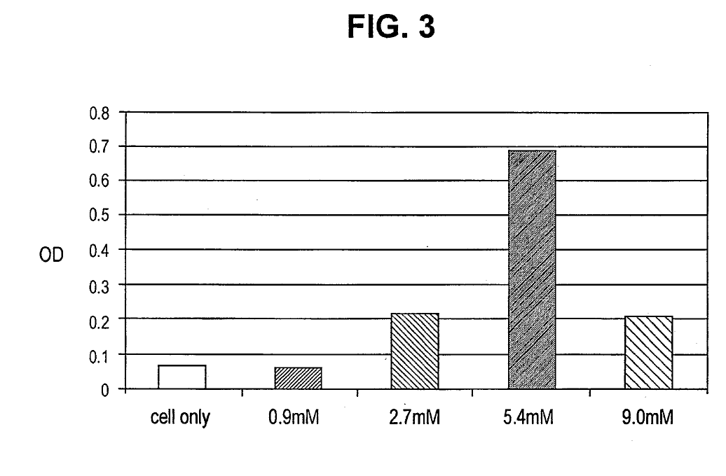

To assess the biological activity of BHT-3021 formulations containing increasing concentrations of calcium a variety of in vitro and in vivo assays may be applied. First, plasmid DNA can be added directly to a transfection competent cell line (e.g. HEK293, HeLa, CHO, etc) and the levels of proinsulin protein produced in the cells can be measured by commercial ELISA (FIG. 3). Second, the different formulations of BHT-3021 can be delivered to mice by IM injection and the quantities of plasmid incorporated into the muscle can be measured at different times post-injection using a BHT-3021 specific quantitative PCR assay (Table 6). Finally, the different formulations can be injected IM at different doses and frequencies and tested in pre-diabetic NOD mice for the ability to prevent the development of autoantibodies, autoreactive T cells, inflammation of the pancreas, and the onset of overt diabetes. Additionally, mice that have already developed hyperglycemia can be treated by injections of the BHT-3021 formulations to determine if the disease can be halted or reversed.

TABLE 6
Muscle plasmid counting analysis following IM injection
of a high calcium formulation of BHT-3021 plasmid DNA.

| | Copies | | | Copies | Average |
|---|---|---|---|---|---|
| | BHT-3021/ | Average | | BHT-3021/ | C_{T} |
| Sample ID | μg DNA | C_{T} Value | Sample ID | μg DNA | Value |
| 2D 1X-1 | >1 × 10⁶ | 16.06 | 2D 6X-1 | NA | 4.51 |
| 2D 1X-2 | >1 × 10⁶ | 16.89 | 2D 6X-2 | NA | 5.90 |
| 2D 1X-3 | >1 × 10⁶ | 17.49 | 2D 6X-3 | NA | 5.36 |
| 2D 1X-4 | >1 × 10⁶ | 17.70 | 2D 6X-4 | NA | 7.17 |
| 7D 1X-1 | 1161 | 29.52 | 7D 6X-1 | NA | 5.42 |
| 7D 1X-2 | 582 | 27.99 | 7D 6X-2 | NA | 6.18 |
| 7D 1X-3 | 1986 | 28.24 | 7D 6X-3 | NA | 5.98 |
| 7D 1X-4 | 422 | 31.28 | 7D 6X-4 | NA | 5.87 |
| 14D 1X-1 | 26899 | 24.74 | 14D 6X-1 | >1 × 10⁶ | 14.50 |
| 14D 1X-2 | 16590 | 25.70 | 14D 6X-2 | >1 × 10⁶ | 16.35 |
| 14D 1X-3 | 297 | 31.74 | 14D 6X-3 | >1 × 10⁶ | 15.66 |
| 14D 1X-4 | 1403 | 29.54 | 14D 6X-4 | NA | 5.73 |

BHT-3021 plasmid was formulated in Dulbecco's PBS with either 0.9 mM calcium chloride (1×) or 5.4 mM calcium chloride (6×). Each formulation was injected into the rear quadriceps muscle of 6 C57B1/6 mice and muscles from 2 mice (n=4 muscles) were harvested at Days 2(2D), 7(7D), and 14(14D) and the number of copies of plasmid in each muscle was quantitated using a BHT-3021 plasmid specific quantitative PCR assay. The injected muscles from the 6× formulation group had much higher levels of plasmid DNA present in the muscles at all time points suggesting the greater stability and persistence of DNA in vivo when formulated with high calcium. Abbreviations: NA—plasmid # too high for quantitation; CT (cycle threshold)—the PCR cycle at which the sample reaches a quantifiable level above assay background.

Although the present invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims that follow. All publications or patent documents cited in this specification are incorporated herein by reference as if each such publication or document was specifically and individually indicated to be incorporated herein by reference. Citation of the above publications or documents is not intended as an admission that any of the foregoing is pertinent prior art, nor does it constitute any admission as to the contents or date of these publications or documents.

What is claimed is:

1. A method of treating an autoimmune disease in a subject associated with one or more self-protein(s), polypeptide(s) or peptide(s) present in the subject non-physiologically comprising administering to the subject: a self-vector comprising an immunosuppressive vector backbone and a polynucleotide encoding the self-protein(s), -polypeptide(s) or -peptide(s) associated with the autoimmune disease; and a divalent cation at a concentration greater than physiological levels.

2. The method of claim 1, wherein the self-vector comprises a BHT-1 vector backbone.

3. The method of claim 1, wherein the autoimmune disease is multiple sclerosis.

4. The method of claim 1, wherein the autoimmune disease is rheumatoid arthritis.

5. The method of claim 1, wherein the autoimmune disease is lupus.

6. The method of claim 1, wherein the self-vector comprises a BHT-1 vector backbone and a polynucleotide encoding human myelin basic protein (MBP).

7. The method of claim 1, wherein the self-vector comprises a BHT-1 vector backbone and a polynucleotide encoding human proteolipid protein (PLP).

8. The method of claim 1, wherein the self-vector comprises a BHT-1 vector backbone and a polynucleotide encoding human myelin associated glycoprotein (MAG).

9. The method of claim 1, wherein the self-vector comprises a BHT-1 vector backbone and a polynucleotide encoding human myelin oligodendrocyte protein (MOG).

10. The method of claim 3, wherein the self-vector is BHT-3009 (SEQ ID NO:3).

11. The method of claim 10, wherein the self-vector BHT-3009 is endotoxin-free.

12. The method of claim 1, wherein the divalent cation is calcium.

13. The method of claim 12, wherein the calcium is at a concentration greater than about 2 mM.

14. The method of claim 12, wherein the calcium is at a concentration of about 5.4 mM.

15. A method of treating multiple sclerosis in a subject comprising administering to the subject a pharmaceutical composition comprising a self-vector comprising an immunosuppressive vector backbone and a divalent cation at a concentration greater than physiological levels.

16. The method of claim 15, wherein the self-vector comprises a BHT-1 vector backbone.

17. The method of claim 15, wherein the self-vector is BHT-3009 (SEQ ID NO:3).

18. The method of claim 17, wherein the pharmaceutical composition is endotoxin-free.

19. The method of claim 15, wherein the divalent cation is calcium.

20. The method of claim 19, wherein the calcium is at a concentration greater than about 2 mM.

21. The method of claim 19, wherein the calcium is at a concentration of about 5.4 mM.

22. A pharmaceutical composition comprising: a self-vector comprising an immunosuppressive vector backbone and a polynucleotide encoding one or more self-protein(s), -polypeptide(s) or -peptide(s) associated with an autoimmune disease; and a divalent cation at a concentration greater than physiological levels.

23. The pharmaceutical composition of claim 22, wherein the self-vector comprises a BHT-1 vector backbone.

24. The pharmaceutical composition of claim 22, wherein the self-vector is BHT-3009 (SEQ ID NO:3).

25. The pharmaceutical composition of claim 22, where in the autoimmune disease is multiple sclerosis.

26. The pharmaceutical composition of claim 22, wherein the autoimmune disease is rheumatoid arthritis.

27. The pharmaceutical composition of claim 22, wherein the autoimmune disease is lupus.

28. The pharmaceutical composition of claim 22, wherein the self-vector comprises a BHT-1 vector backbone and a polynucleotide encoding human myelin basic protein (MBP).

29. The pharmaceutical composition of claim 22, wherein the self-vector comprises a BHT-1 vector backbone and a polynucleotide encoding human proteolipid protein (PLP).

30. The pharmaceutical composition of claim 22, wherein the self-vector comprises a BHT-1 vector backbone and a polynucleotide encoding human myelin associated glycoprotein (MAG).

31. The pharmaceutical composition of claim 22, wherein the self-vector comprises a BHT-1 vector backbone and a polynucleotide encoding human myelin oligodendrocyte protein (MOG).

32. The pharmaceutical composition of claim 25, wherein the self-vector is BHT-3009 (SEQ ID NO:3).

33. The pharmaceutical composition of claim 32, wherein the pharmaceutical composition is endotoxin-free.

34. The pharmaceutical composition of claim 22, wherein the divalent cation is calcium.

35. The pharmaceutical composition of claim 34, wherein the calcium is at a concentration greater than about 2 mM.

36. The pharmaceutical composition of claim 34, wherein the calcium is at a concentration of about 5.4 mM.

37. A pharmaceutical composition comprising BHT-3009 (SEQ ID NO:3) and a divalent cation at a concentration greater than physiological levels.

38. The pharmaceutical composition of claim 37, wherein BHT-3009 is endotoxin-free.

39. A self-vector BHT3009 (SEQ ID NO:3).


SEQUENCE LISTING (12 sequences)

SEQ ID NO: 1 - DNA, 2998 bp, Artificial Sequence
  synthetic modified pVAX1 self-vector
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 2 - DNA, 2998 bp, Artificial Sequence
  synthetic self-vector BHT-1 expression vector backbone with cytosine to non-cytosine substitutions
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggcggctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgcggccaaggatctgatggcgcaggggatcaagctctgatcaagagacaggatgaggatggtttcgcatgattgaacaagatggattgcacgcaggttctccggcagcttgggtggagaggctattcggctatgactgggcacaacagacaatcggctgctctgatgccgccgtgttcaggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggcaggttttctggattcatcgactgtggccggctgggtgtggcggacaggtatcaggacatagcgttggctacccgtgatattgctgaagagcttggcggcgaatgggctgacaggttcctcgtgctttacggtattgcggctcccgattcgcagcgcattgccttctataggcttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 3 - DNA, 3485 bp, Artificial Sequence
  synthetic self-vector BHT-3009, BHT-1 expression vector backbone with cytosine to non-cytosine substitutions and polynucleotide encoding full-length human myelin basic protein (MBP, hMBP) insert
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgtgatggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgcccaagcggggctctggcaaggactcacaccacccggcaagaactgctcactacggctccctgccccagaagtcacacggccggacccaagatgaaaaccccgtagtccacttcttcaagaacattgtgacgcctcgcacaccacccccgtcgcagggaaaggggagaggactgtccctgagcagatttagctggggggccgaaggccagagaccaggatttggctacggaggcagagcgtccgactataaatcggctcacaagggattcaagggagtcgatgcccagggcacgctttccaaaatttttaagctgggaggaagagatagtcgctctggatcacccatggctagacgctgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgcggccaaggatctgatggcgcaggggatcaagctctgatcaagagacaggatgaggatggtttcgcatgattgaacaagatggattgcacgcaggttctccggcagcttgggtggagaggctattcggctatgactgggcacaacagacaatcggctgctctgatgccgccgtgttcaggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggcaggttttctggattcatcgactgtggccggctgggtgtggcggacaggtatcaggacatagcgttggctacccgtgatattgctgaagagcttggcggcgaatgggctgacaggttcctcgtgctttacggtattgcggctcccgattcgcagcgcattgccttctataggcttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 4 - DNA, 22 bp, Artificial Sequence
  synthetic immune modulatory sequence (IMS) immunostimulatory sequence (ISS) with core hexamer and flanking sequences
tgactgtgrrcgyyagagatga

SEQ ID NO: 5 - DNA, 23 bp, Artificial Sequence
  synthetic immune modulatory sequence (IMS) immunoinhibotory sequence (IIS) with core hexamer and flanking sequences
ttgactgtgrynnyyagagatga

SEQ ID NO: 6 - DNA, 30 bp, Artificial Sequence
  synthetic poly G region flanking immune modulatory sequence (IMS)
gggggggggggggggggggggggggggggg

SEQ ID NO: 7 - DNA, 16 bp, Artificial Sequence
  synthetic immune modulatory sequence (IMS) with polyG region two nucleotides 3′ from core hexamer
ccatgtggttatgggt

SEQ ID NO: 8 - DNA, 120 bp, Artificial Sequence
  synthetic immune modulatory sequence (IMS) suppressive oligonucleotide forming G-tetrad
ttagggttagggttagggttagggttagggttagggttagggttagggttagggttagggttagggttagggttagggttagggttagggttagggttagggttagggttagggttaggg

SEQ ID NO: 9 - DNA, 800 bp, Artificial Sequence
  synthetic immune modulatory sequence (IMS) suppressive oligonucleotide
tgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggttgggcggt

SEQ ID NO: 10 - DNA, 22 bp, Artificial Sequence
  synthetic immune modulatory sequence (IMS) suppressive oligonucleotide
gggtgggtgggtattaccatta

SEQ ID NO: 11 - DNA, 22 bp, Artificial Sequence
  synthetic immune modulatory sequence (IMS) suppressive oligonucleotide
ttagggttagggtcaaccttca

SEQ ID NO: 12 - DNA, 22 bp, Artificial Sequence
  synthetic immune modulatory sequence (IMS) suppressive oligonucleotide
gggsaagctggaccttgggggg